(19) United States
(12) Patent Application Publication
(10) Pub. No.: US 2025/0143589 A1
(43) Pub. Date: May 8, 2025

(54) COMPACT LASER-POWERED SPECKLE VISIBILITY SPECTROSCOPY DEVICES

(71) Applicant: California Institute of Technology, Pasadena, CA (US)

(72) Inventors: Simon Mahler, Pasadena, CA (US); Yu Xi Huang, South Pasadena, CA (US); Changhuei Yang, South Pasadena, CA (US)

(21) Appl. No.: 18/935,159

(22) Filed: Nov. 1, 2024

Related U.S. Application Data
(60) Provisional application No. 63/547,269, filed on Nov. 3, 2023.

(51) Int. Cl. A61B 5/0205; A61B 5/00; A61B 5/02; A61B 5/024; A61B 5/0255; A61B 5/026

(52) U.S. Cl. CPC A61B 5/0205; A61B 5/02028; A61B 5/0261; A61B 5/4064; A61B 5/6803; A61B 5/7271; A61B 5/02438; A61B 5/02444; A61B 5/0255; A61B 2560/0214; A61B 2560/0462; A61B 2576/026

(57) ABSTRACT

Techniques for monitoring cerebral blood metrics such as cerebral blood flow, cerebral blood volume and/or heart rate are provided. In some embodiments, techniques involve a headset with at least one laser configured to emit light into the brain and one or more light detectors such as CMOS sensors that generate information indicative of light reflected from one or more structures within the brain, which can be used to determine the cerebral blood metrics. Each light detector may be positioned within 5 mm of the scalp in some cases.

### CROSS-REFERENCES TO RELATED APPLICATION

This application claims priority to and benefit of U.S. Provisional Patent Application No. 63/547,269, titled “Compact and Cost-Effective Laser-Powered SVS Device for Assessing Cerebral Blood Flow and Cerebrovascular Reactivity,” filed on Nov. 3, 2023, which is hereby incorporated by reference in its entirety and for all purposes.

### FEDERALLY SPONSORED RESEARCH OR DEVELOPMENT

This invention was made with government support under Grant No. EY033086 awarded by the National Institutes of Health. The government has certain rights in the invention.

### FIELD

Certain aspects generally relate to techniques for monitoring blood metrics, and more specifically, to methods and apparatus for determining cerebral blood flow, cerebral blood volume, and/or heart rate.

### BACKGROUND

The brain stands as the most complicated and indispensable organ within the human body. Yet today, despite significant advancements, the brain is the organ for which our understanding lags behind other vital organs. Many aspects of the brain's functioning remain a subject of ongoing research and discovery. The brain is highly complex and plays a significant role in overseeing vital bodily functions such as the rate of oxygenation and blood circulation. It also regulates its own blood supply to ensure a consistent delivery of oxygen and nutrients. Together, these mechanisms guarantee the maintenance of physiological conditions within a precise and optimal range. Thus, monitoring cerebral blood flow (CBF) non-invasively bears significance in both clinical settings and cognitive neuroscience research.

### SUMMARY

Techniques disclosed herein may be practiced with a processor-implemented method, an apparatus comprising one or more processors and one or more processor-readable media, and/or one or more non-transitory processor-readable media.

Certain embodiments pertain to headsets or other apparatus for generating cerebral blood metric data. In some embodiments, a headset for generating cerebral blood metric data includes a headband configured to be worn on a head, a laser coupled to the headband and configured to emit light into a brain within a skull of the head, a light detector coupled to the headband and configured to generate information indicative of light reflected from one or more structures within the brain, wherein the light detector is configured to contact or be within 5 mm of a scalp of the skull, and a power source coupled to the headband and in electrical communication with the laser.

In some embodiments, a headset may be free of optical fibers.

In some embodiments, an apparatus has a low profile.

In some embodiments, the headset is a multi-channel headset including a headband configured to be worn on a head during operation; and a plurality of channels coupled to the headband. Each includes a laser configured to emit light into a brain within a skull of the head, a light detector configured to generate information indicative of light emitted by the laser and reflected by one or more structures within the brain, and a power source in electrical communication with the laser. The light detectors of the channels are configured to probe different regions of the brain.

In some embodiments, an apparatus includes a circuit board, one or more processors attached to the circuit board, a laser diode attached to the circuit board, a light detector in electrical communication with the one or more processors (e.g. an application-specific integrated circuit or a field programmable gate array), and a light block located between the laser diode and the light detector (e.g., one or more CMOS sensors). A power source may be in electrical communication with the laser diode. In some cases, the apparatus also includes a wireless transmitter. One or more of these apparatus may be attached to a headband according to an implementation.

Certain embodiments pertain to methods for generating cerebral blood metric data. In some embodiments, a method includes causing, using a laser, light to be emitted into a brain within a skull of a head of a user, obtaining, using a light detector, information indicative of light reflected from one more structures within the brain, and based on the obtained information, determining one or more cerebral blood metrics as a function of time. In some cases, the method may also include normalizing each speckle image based on a first set of the speckle images acquired during a first time period, calculating a speckle contrast of each normalized speckle image, and adjusting the speckle contrast to account for noise.

These and other features are described in more detail below with reference to the associated drawings.

### BRIEF DESCRIPTION OF THE DRAWINGS

Certain aspects are illustrated by way of example and not limitation in the figures of the accompanying drawings in which like references indicate similar elements.

FIG. 1 depicts a schematic illustration of a front view of components of an apparatus for generating cerebral blood metrics data, according to some embodiments.

FIG. 2 depicts a schematic illustration of a top view of the apparatus in FIG. 1 illustrating example light paths through the skull and a portion of a brain of a user, according to some embodiments.

FIG. 3A depicts a drawing of an isometric view of a light emission package, according to some embodiments.

FIG. 3B depicts a drawing of an exploded view of the light emission package in FIG. 3A.

FIG. 4A depicts a drawing of an isometric view of a detection package, according to some embodiments.

FIG. 4B depicts a drawing of an exploded view of the detection package in FIG. 4A.

FIG. 5A depicts a drawing of an isometric view of a detection package having a heat sink, according to some embodiments.

FIG. 5B depicts a drawing of the heat sink in FIG. 4A.

FIG. 6 depicts a drawing of an exploded view of the detection package in FIG. 5A.

FIG. 7A depicts a schematic illustration of an isometric view of a multi-channel apparatus for generating cerebral blood metrics data, according to some embodiments.

FIG. 7B depicts a schematic illustration of a top view of the multi-channel apparatus in FIG. 7A, according to some embodiments.

FIG. 8A depicts a schematic illustration of the computing system of the multi-channel apparatus in FIG. 7A, according to some embodiments.

FIG. 8B depicts plots of traces of cerebral blow flow and cerebral blood volume metrics measured over time by the multi-channel apparatus in FIG. 7A, according to some embodiments.

FIG. 9A depicts a schematic illustration of an isometric view of an apparatus for generating cerebral blood metrics data, according to some embodiments.

FIG. 9B depicts a schematic illustration of a top view of the apparatus for generating cerebral blood metrics data in FIG. 9A, according to some embodiments.

FIG. 9C depicts a schematic illustration of a side view of the apparatus for generating cerebral blood metrics data in FIG. 9A, according to some embodiments.

FIG. 10 depicts a schematic illustration of an example of a process flow for determining cerebral blood flow from raw images, according to embodiments.

FIG. 11 depicts a schematic illustration of an example of a process flow for determining cerebral blood flow from raw images, according to embodiments.

FIG. 12 depicts a flowchart of an example method of determining cerebral blood metrics, according to embodiments.

FIG. 13 depicts images illustrating a normalization operation of a method of determining cerebral blood metrics, according to embodiments.

FIG. 14 depicts example graphs of cerebral blood metric data based on different source-to-detector (S-D) distances for a subject, in accordance with embodiments.

FIG. 15 depicts example graphs of cerebral blood metric data based on different source-to-detector (S-D) distances for ten subjects, in accordance with embodiments.

FIG. 16 depicts example graphs of cerebral blood flow during a breath hold task in accordance with some embodiments.

FIG. 17 depicts example graphs of cerebral blood flow during a breath holding task in accordance with some embodiments.

FIG. 18 depicts example graphs of cerebral blood flow and cerebral blood volume during a breath holding task in accordance with some embodiments.

FIG. 19 depicts a diagram of components of an example computing device in accordance with some embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Tracking cerebral blood flow non-invasively is essential but challenging when evaluating cerebral autoregulation and detecting cerebrovascular irregularities. While ultrasound and magnetic resonance imaging (MRI) have been used to measure cerebral blood flow, optical methods may be advantageous due to their high sensitivity and temporal resolution. For example, optical techniques are more sensitive than ultrasound methods since the wavelength of light being used is at least an order of magnitude smaller than the typical ultrasound wavelength. In addition, optical techniques may provide higher temporal resolution (e.g., 50-100 Hz) than MRI methods.

Speckle visibility spectroscopy (SVS) (also referred to as “speckle contrast optical spectroscopy” (SCOS)) is a technique that can be used to infer blood flow from the spatial ensemble of speckle fields in images captured by a camera. In SVS, tissues being probed are illuminated and photons reflected from structures in the tissues are collected by pixels of the camera within the same frame. The camera typically acquires images with an exposure time longer than the decorrelation time of the speckle field. This results in multiple different speckle patterns being summed up into a single camera frame. As the speckle field fluctuates, each speckle pattern recorded by the camera is smeared and washed out within the exposure time. The smearing or washing out is due to the dynamics of the blood cells. A decorrelation time can be calculated from the degree of blurring of the captured frame, typically by calculating the speckle contrast. Blood flow may be determined from the speckle contrast.

Disclosed herein are techniques that use SVS to monitor blood metrics. In particular, certain techniques pertain to apparatus that use SVS to monitor cerebral blood flow metrics including cerebral blood volume (CBV), cerebral blood flow (CBF), and/or heart rate. These apparatus are also referred to as SVS apparatus or SVS devices. The data used to determine cerebral blood metrics may be obtained during a period of time that includes time a user such as a patient is holding their breath (sometimes referred to as a “breath-holding time period”) or a period of time before or after the user is holding their breath. For example, normalization data may be obtained during a baseline time period before the breath-holding time period.

Cerebral blood metrics may be determined using data characterizing light reflected from various structures within a brain of the user. For example, in some implementations, cerebral blood volume, cerebral blood flow and/or heart rate metrics may be determined by transmitting light in a near-infrared or infrared wavelength into the brain of the user and measuring reflected light. In some implementations, cerebral blood flow may be determined from the speckle contrast in images based on the reflected light from the brain using SVS.

In certain implementations, an apparatus includes a laser (e.g. laser diode) or other light source and a light detector, which may be a camera, or the like. The light detector may include one or more sensors (e.g., one or more CMOS sensors). The light source and light detector may be attached to a headband or other structure that can encircle the head of a user such as, e.g., around the forehead. Light may be transmitted through the skull into the brain using the light source. Reflected light from one or more structures in the brain may be captured using the light detector. The light detector is configured in the headband to be in close proximity to, or in direct contact with, the scalp when the headband is encircling the head. In some cases, the headband may be tightened to apply pressure to the scalp via contact with the light detector to reduce blood flow locally. Placing the light detector as close as possible to skull maximizes the numerical aperture of the optical imaging system and number of photons received by the light detector. This configuration is also advantageous over optical systems with collection optical fiber between the scalp and the light detectors. For example, optical systems relying on fiber collection can be susceptible to fluctuations caused by the movement of collection fiber which affects speckle dynamics. By placing the light detector in direct contact with the scalp surface, this source of instability is avoided, and more consistent results are achieved with a simpler arrangement. In one example, the apparatus can achieve a sampling rate of 50 Hz while being minimally affected by external disturbances.

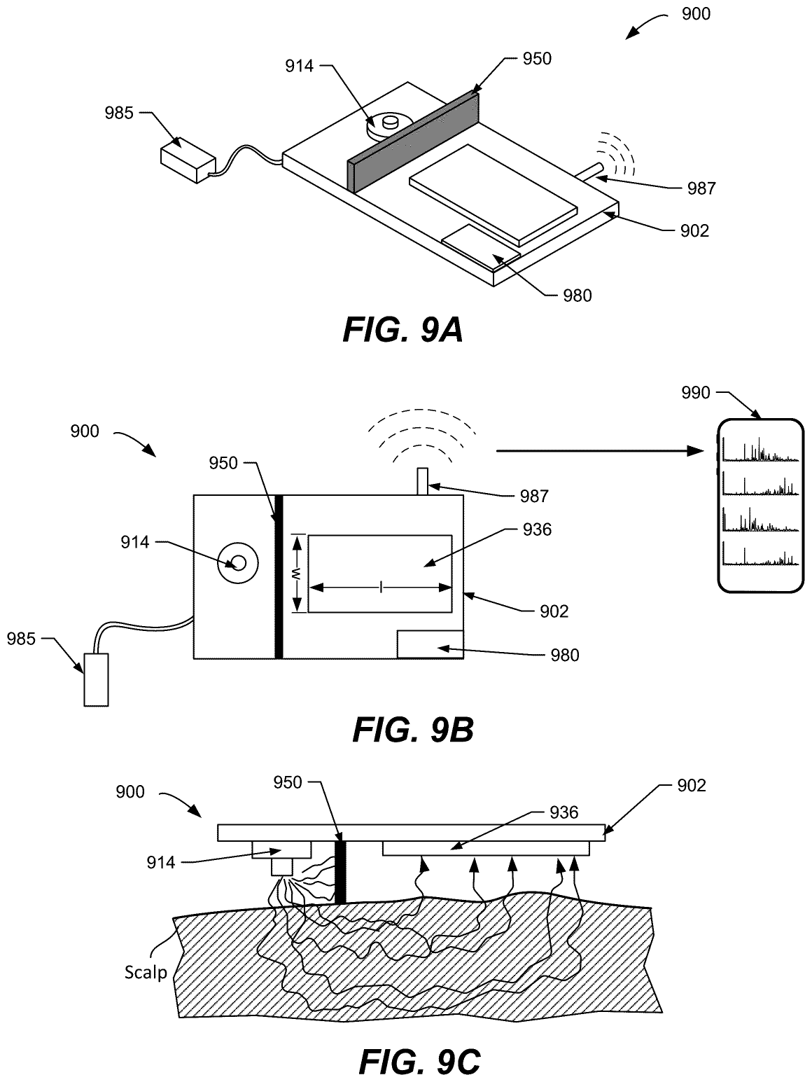

Certain implementations pertain to compact apparatus for non-invasively monitoring cerebral blood flow (CBF) and cerebrovascular reactivity. In some cases, the compact apparatus includes a light source such a laser diode and a light detector such as a CMOS board camera that are in a single package that can be placed on a head to measure CBF. An example of components of such an apparatus are shown in FIGS. 9A-9C. In some instances, these apparatus may provide real-time CBF monitoring at, for example, a 50 Hz sampling rate. These apparatus may also provide the advantages of being lightweight and low cost.

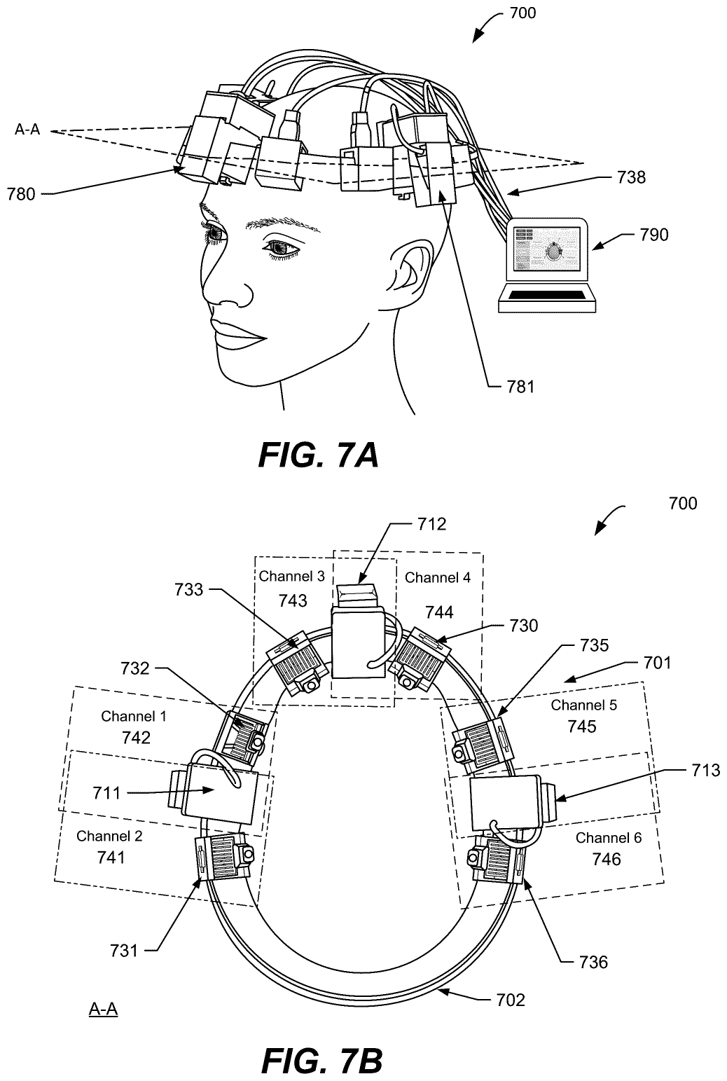

In some embodiments, an apparatus includes multiple light sources and multiple light detectors. For example, an apparatus may be a multi-channel apparatus with multiple light source and light detector (source-detector) pairs forming respective channels such as the multi-channel apparatus 700 with six channels 741, 742, 743, 744, 745, and 746 shown in FIGS. 7A-B. Generally speaking, a multi-channel apparatus may have any suitable number of channels (e.g., 2, four, six, eight, ten, etc.) disposed at various locations to be able to probe different regions of the brain simultaneously or at different times. In some instances, two or more channels may share the same light source. For example, as shown in FIG. 7B, channel 1 741 shares a light source 730 with channel 2 742.

In some embodiments, one or more light sources (sometimes referred to herein as “sources”) are packaged as a light emission package and a light detector (sometimes referred to herein as a “detector”) is packaged as a light detection package. In some implementations, one or more light emission packages and one or more light detection packages may be coupled to a headband or cap at different positions or may be adjustable to move along the headband or cap to different positions to be able to take simultaneous measurements of different regions of the brain such the parietal lobe, the frontal lobe, etc. A light source may be coupled to the headband or cap via the light emission package and a light detector may be coupled to the headband or cap via the light detector package.

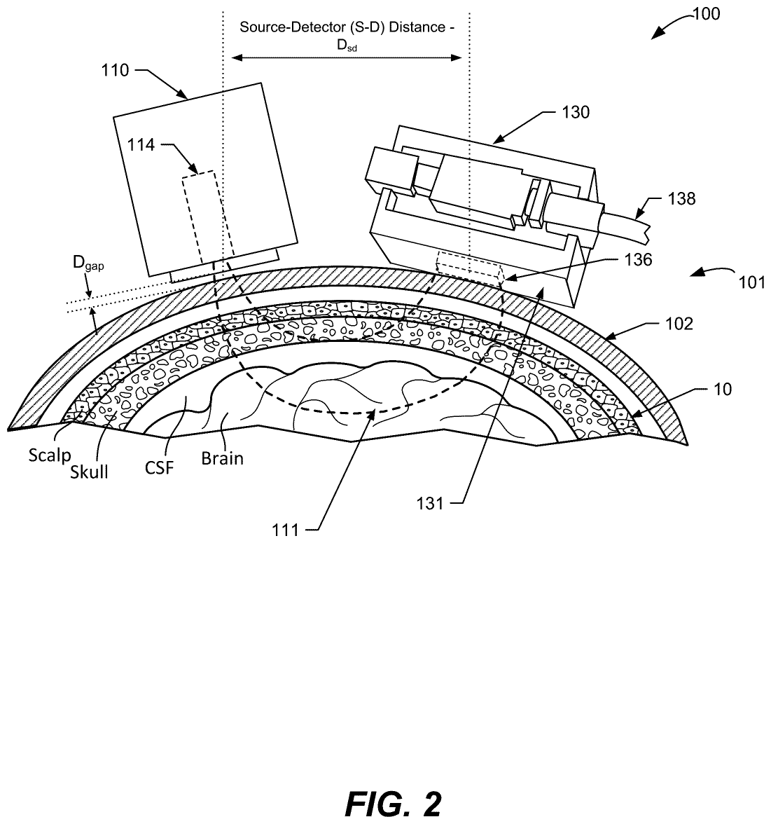

The distance between the illumination spot of the light source and a corresponding light detector is generally referred to as the “source-detector distance,” or “S-D distance” and example values may include 2 cm, 2.5 cm, 3 cm, 3.5 cm, 4 cm, etc. In some implementations, the S-D distance is within a range of about 3.0 cm-4 cm. In some implementations, the S-D distance is within a range of about 2.5 cm-3.5 cm. In some implementations, the S-D distance is within a range of about 3.0 cm-5.0 cm. An S-D distance of between 3.0 cm and 4.0 cm may provide the advantage that the majority of collected photons have traveled through portions of the brain which may provide the high brain specificity needed to detect cerebral blood flow. In instances in which a headset includes multiple channels, the distance between the light detector and the corresponding light source for different channels may be different. In some implementations, positions of light sources and/or light detectors may be modifiable. For example, a light emission package and/or a light detection package may be adjustable in position via screws, Velcro, or other similar adjustable hardware. This may allow for S-D distances to be modified, which may in turn allow the depth of imaging to be modified as different source-detector distances impact the depth the emitted light can penetrate within the brain, as shown in and described below in connection with FIG. 2. Additionally, modification of positions of light sources and/or light detectors may allow different brain regions to be probed simultaneously using one headset.

In some embodiments, an apparatus may have a light source and light detector coupled to a single circuit board, which is sometimes referred to herein as a combined source-detector package. In some cases, this apparatus includes one or more processing units or processors (e.g., an application-specific integrated circuit (ASIC) or a programmable logic device such as a field-programmable gate array (FPGA)), a laser diode, and a light detector coupled to the circuit board. The apparatus also includes a light block (e.g., a wall of opaque material) located between the laser diode and the light detector to avoid cross talk. The apparatus also includes a power source in electrical communication with the laser diode. An example of such an apparatus is apparatus 800 shown in FIGS. 9A-9C, which is described in more detail below. In some cases, the light detector may be segmented. For example, the light detector may include multiple sensors arranged at different locations at different S-D distances from the laser diode to probe different regions. In these cases, the apparatus includes multiple channels corresponding to the light source and different detector segments.

In some implementations, the light detector package may be fiber free in that there is no optical fiber coupling to the light detector. In these cases, the light detector may be placed directly atop the user's scalp. In addition or alternatively, the light emission package may be fiber free since the light source is an integral part of the light emission package and does not required optical fiber to communicate the light from an external source. In various embodiments, all components of a headset may be fiber free, which avoids noise emanating from optical fibers.

In addition to measuring cerebral blood flow, the apparatus of various embodiments can measure cerebral blood volume and heart rate, allowing the simultaneous measurement of multiple cerebral blood metrics at single or multiple regions of the brain. With its cost-effectiveness, scalability, versatility, and user-friendliness, the laser-based apparatus of various examples hold great promise for advancing non-invasive cerebral monitoring technologies.

In some embodiments, a light detector may be divided into segments where the segments can simultaneously generate data based on light collected from different regions of the brain. Segmentation allows data for different depths (corresponding to different source-detector distances) to be measured simultaneously using a single light detector. Segmentation may also help calibrate noise and provide redundancy by effectively using one light detector as multiple image generating sources. For example, the light detector may be composed of multiple sensors such as multiple CMOS sensors at different locations that can capture frames from different regions of the brain simultaneously. In some cases, the light detector may include multiple CMOS sensors arranged at different S-D distances from the light source along a length to capture frames of regions at different depths within the brain. As another example, the light detector may include one sensor with a two-dimensional array of pixels where the array is divided into different sets (segments) of pixels at different locations.

In cerebrovascular monitoring, primary metrics typically include blood pressure, which influences cerebral blood flow, and the cerebral blood volume, which is contingent upon vessel radius. In typical cerebral autoregulation situations, the autoregulation system attempts to maintain a constant blood pressure by modulating the blood vessel's diameter, which is measured by blood volume. However, the extent to which blood vessels can be dilated is limited and there exist some breakdown regime where vessels can no longer dilate to modulate the increasing blood pressure. In some implementations, the apparatus or a separate computing device may detect a breakdown zone from the blood flow metrics. Ideally, when the breakdown zone is detected, further physiological tests will be conducted.

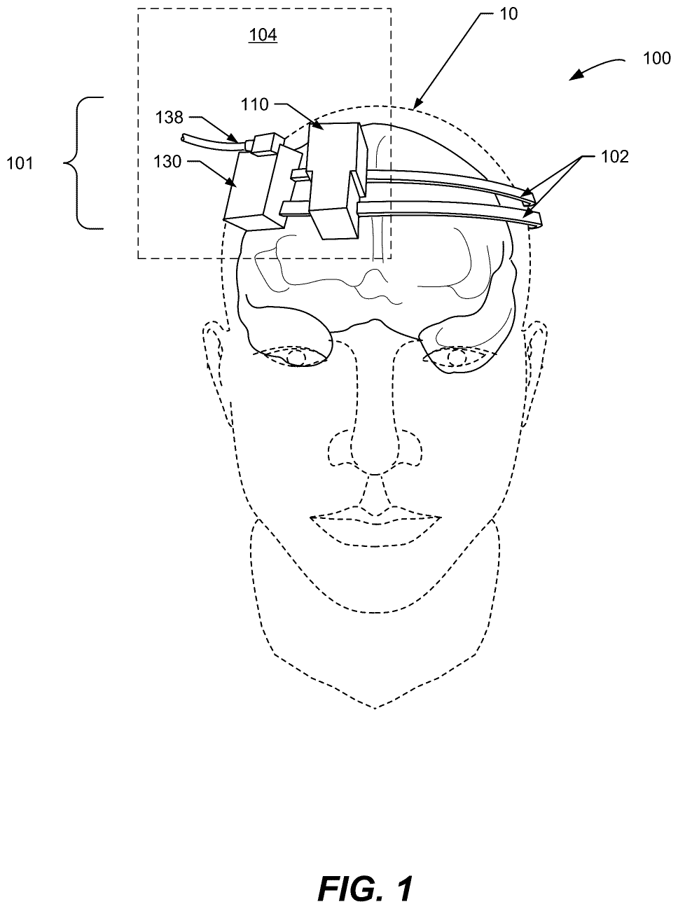

FIG. 1 depicts a schematic drawing of a front view of an example of an apparatus 100 for generating cerebral blood metrics data using SVS, according to various embodiments. FIG. 2 is a schematic diagram of a top view of the apparatus 100 in FIG. 1. In the illustrated example, the apparatus 100 includes a headset 101 with a headband 102. The headset 101 includes a light emission package 110 having a light source 114 such as a laser and a light detector package 130 having a light detector 136 (e.g. a CMOS board camera). The light emission package 110 and light detector package 130 form a channel 104. The light emission package 110 and light detector package 130 are coupled to a portion of the headset 101. In one implementation, the light emission package 110 and light detector package 130 are adjustable to different positions along the headband 102. As shown, the headband 102 is configured to encircle at least a portion of a head 10 of a user at, e.g., the forehead level.

The apparatus 100 also includes an electrical connection 138 (e.g., USB cable) between the light detector 136 of the light detector package 130 and one or more processors and/or a computing device (e.g., computing device 790 in FIG. 7A) for communicating data. In other implementations, apparatus 100 may omit electrical connection 138 and includes a wireless transmitter for communicating data to a wireless receiver in electrical communication with the one or more processors and/or computer readable medium. The processor(s) or computing device may perform operations to determine one or more cerebral blood metrics based on the data captured by the light detector.

In various embodiments, an apparatus for generating cerebral blood metrics data includes a headset with a headband configured to be worn on a head of a user during operation. The headband may include a strap or a cap that can encircle the head of the user. The headband may include a tightening mechanism such as Velcro, latch, or the like that can secure and tighten the headband to the head. In some cases, the tightening of the headband may be used to place the light detector in contact with and/or apply pressure to the scalp locally where the light detector contacts the scalp. Contact pressure may be desirable to reduce blood flow at the scalp locally to decrease the scalp's influence and increase brain specificity. In addition, the headband, the one or more light detectors, and/or more or more light sources may be configured to allow adjustment of the locations of the light detector(s) and light source(s) to probe different regions/depths of the brain.

According to various embodiments, a headset may include one or more channels (e.g., one, two, three, four, five, six, seven, eight, nine, ten, etc.). For example, as shown in FIGS. 7A and 7B, the headset 701 includes six channels, 741, 742, 743, 744, 745, and 746. As another example, a headset may include four channels. By way of example, four channels may be implemented to measure cerebral blood metrics at a frontal lobe region, a parietal lobe region, or the like.

Referring back to FIG. 2, channel 104 includes a light source 114 and a light detector 136 (e.g., a camera such as a board camera). A first surface 131 of the detector package 130 is shown in contact with the scalp during operation. In some cases, the headband 102 may be tightened to apply a pressure to the scalp at a region where the detector package 130 contacts the scalp to decrease the blood flow in the scalp locally. The light source 114 may be a laser configured to emit coherent laser light in a near-infrared or infrared wavelength into the head 10 of the user. In one example, the laser may emit light at about 830 nm. In another example, the laser may emit light at about 785 nm. The light detector 136 may include one or more CMOS sensors or may be a photodiode. There is a gap between the light emission package 110 and the scalp with a gap distance, D_{gap}. In some cases, the gap distance, D_{gap}, may be in a range of 5 mm and 7 mm. In one example, the gap distance, D_{gap}, may be set to generate an illumination spot with the given light intensity level that lies within safety standards.

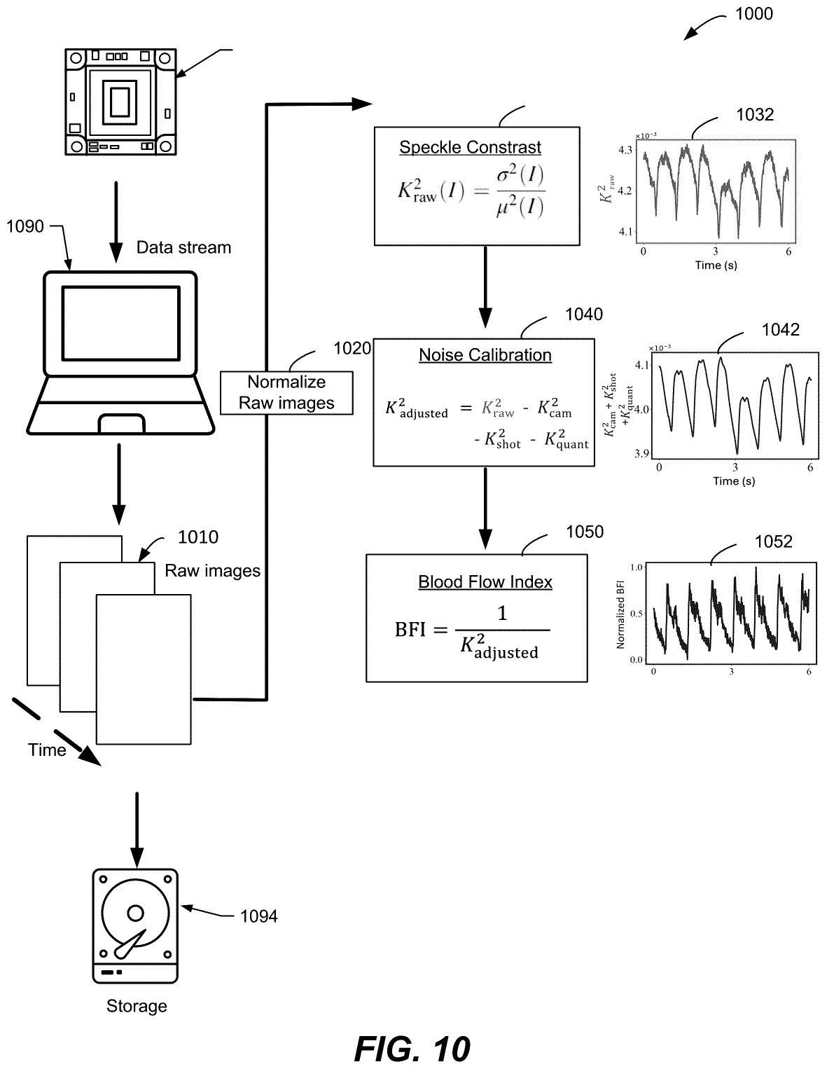

As illustrated, the light detector 136 may obtain light reflected off various brain structures from light emitted by light source 114 as illustrated by the example light paths 111. Data captured by the light detector 136 may be used to determine cerebral blood metrics. Some examples of operations that may be implemented to determine one or more cerebral blood metrics are described below in connection with FIGS. 10, 11, and 12.

Returning to FIG. 2, the illumination spot of the light source 114 and the light detector 136 are at a source-detector distance (S-D distance), D_{sd}, from each other. The S-D distance impacts the depth that the emitted light can penetrate into the brain. In certain implementations, the S-D distance may be adjusted to tune the depth of penetration into the brain. The locations of the light detector 136 and light source 114 determine the region being imaged. In the illustration, the light detector 136 is positioned in contact with the scalp and at the S-D distance from the illumination spot of the light source 114 in order to collect light emerging from the brain at a distance away from the laser illumination spot. In some cases, the sensor area of the light detector 136 may in the range of 25 mm² and 100 mm² and the pixel pitch may be in a range of 2 μm and 4 μm. The spatial distribution of the exiting photons collected by the light detector 136 exhibits a granular pattern in the images with areas of high and low intensity, which are referred to as “speckles.” The light detector 136 is typically configured with an exposure time that is significantly larger than the decorrelation time, Tc, of the speckle field. For example, exposure times may be in a range of 1 ms and 10 ms. This results in multiple speckle patterns integrated into a single camera frame. The motions within the light paths 111 are primarily due to movement of blood cells and will scatter and change the effective optical path lengths resulting in a fluctuating speckle field that varies in time. The speckle contrast calculated from the images can be used to determine cerebral blood flow.

In various embodiments, an apparatus for generating cerebral blood metric data includes a headset with one or more light emission packages and one or more light detection packages coupled to a headband or cap. Each light emission package includes at least one light source and each light detection package includes a light detector. In one example, the light emission package may include a plurality of light sources such as multiple laser diodes configured to emit light at different wavelengths. In other embodiments, an apparatus includes a headset with one or more combined source-detector packages. An example of a combined source-detector package is described below in connection with FIGS. 9A, 9B, and 9C.

In various embodiments, an apparatus for generating cerebral blood metric data includes a light source that is a laser (e.g. a laser diode). The laser may be a continuous wave laser. The laser may be configured to emit light in a near-infrared or infrared wavelength. In one example, the laser may emit light at about 830 nm. In another example, the laser may emit light at about 785 nm. In one instance, the light source may be a single-mode continuous wave 785 nm laser diode (e.g., Thorlabs laser L785H1) that can deliver up to 200 mW. In other implementations, other wavelength may be used such as 1064 nm. The light emission package may include a housing (e.g., a 3-D printed mount) within which the light source may be mounted. The light source may be placed at a predetermined distance, D_{gap}, (e.g., 6 mm) from the scalp to generate a spot diameter of 5 mm or larger during operation.

In various embodiments, an apparatus for generating cerebral blood metric data includes a light detector, which may be a camera, or the like. A light detector may be positioned to collect light from the brain and output a data signal including data representative of one or more image frames captured over time. Each image frame may include an intensity distribution of light received at a sensing region. The light detector is configured to operate at a frame rate (e.g., 50 frames per second (fps)) and has a pixel pitch (e.g., 3.4 μm). The light detector may include one or more sensors. Some examples of suitable sensors include a complementary metal-oxide-semiconductor (CMOS) sensor, a linear or array charge-coupled device (CCD), and other similar devices. In one embodiment, the sensor may be a CMOS sensor in a flexible format such as the Cappella CMOS image sensor sold by Teledyne. In one embodiment, the light detector is a USB-Board camera (e.g., the Basler daA1920-160 μm camera with a Sony Sensor IMX392). In some cases, the light detector package may include a housing (e.g., a 3-D printed mount) within which the light detector may be mounted. The housing may be employed to prevent laser light reflections and stray light. In some cases, the light detector may include one or more CMOS sensors and have a width in a range of 5 mm and 10 mm and a length in a range of 5 mm and 10 mm. In some cases, the pixels of a CMOS sensor may have a size in a range of 2 μm and 4 μm.

According to one aspect, a light detector may include multiple segments. This segmentation allows data from multiple source-detector distances to be measured simultaneously with a single light detector. For example, the light detector may include multiple sensors such as multiple CMOS sensors. The CMOS sensors may be arranged at different S-D distances from the light source such that the frames are captured of regions at different depths. As another example, the light detector may include one sensor with different sets (segments) of pixels at different locations.

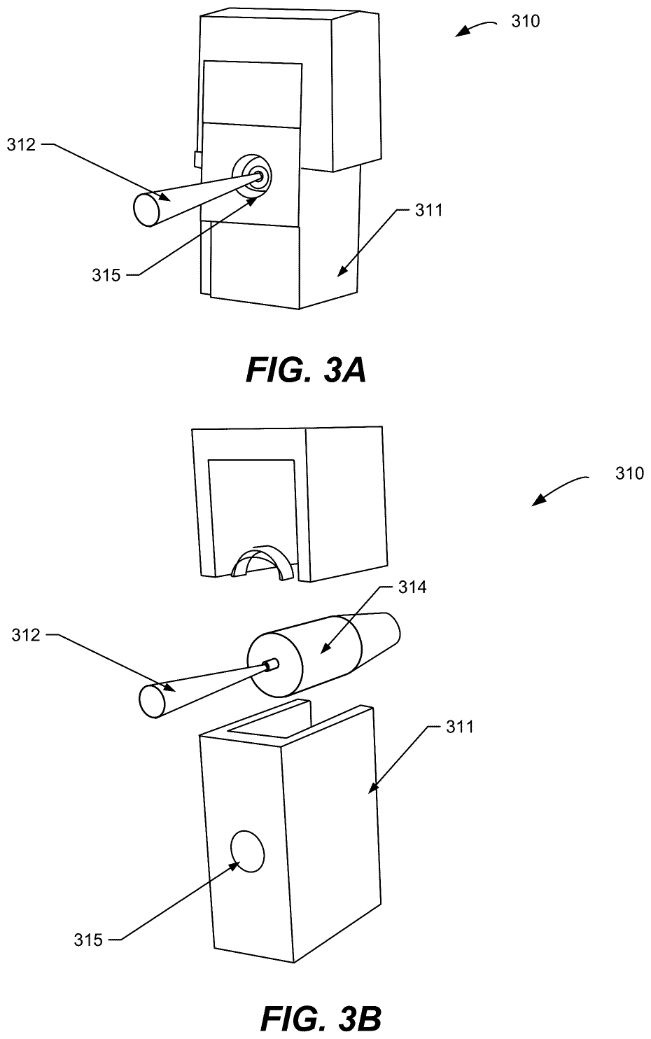

FIG. 3A is a drawing of an isometric view of a light emission package 310, according to some embodiments. FIG. 3B is a drawing of an exploded view of the light emission package 310 in FIG. 3A. The light emission package 310 includes a housing 311 having an aperture 315. The light emission package 310 also includes a laser 314 mounted within the housing 311. The laser 314 is configured to emit a laser beam 312 through the aperture 315 in the housing 311. The components of light emission package 310 are examples of components that can be implemented in the light emission package 110 of apparatus 100 in FIG. 1 and the light emission packages of the multi-channel apparatus 700 in FIGS. 7A and 7B.

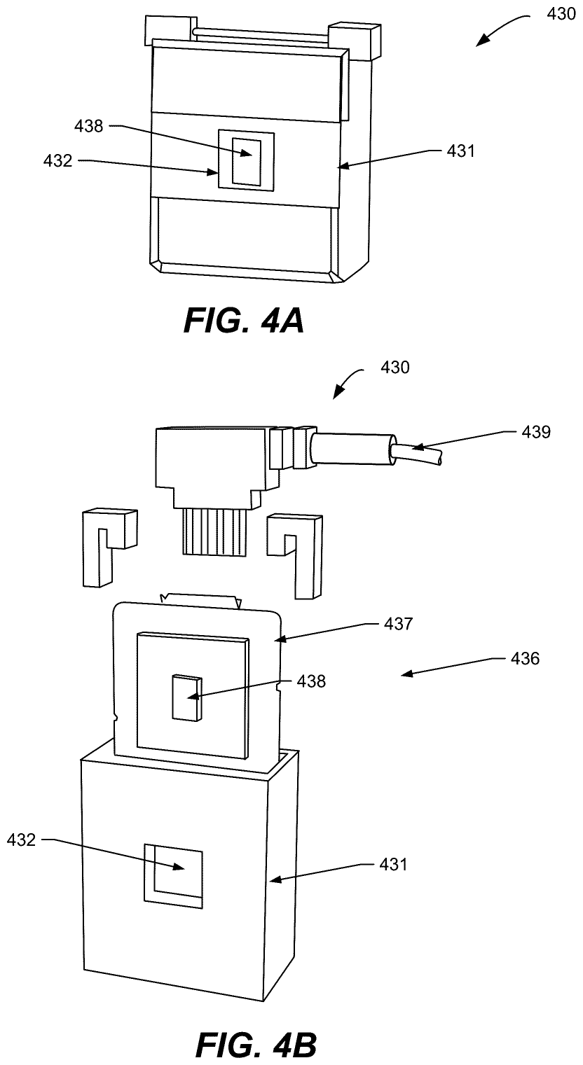

FIG. 4A is a drawing of an isometric view of a detection package 430, according to some embodiments. FIG. 4B is a drawing of an exploded view of the detection package 430 in FIG. 4A. The detection package 410 includes a housing 411 having an aperture 432. The detection package 410 also includes a light detector 436 in the form of a USB-board camera (e.g., Basler daA3840-45 μm camera board) having a CMOS sensor 438 (e.g., Sony IMX392 CMOS sensor). The frame speed of the USB-board camera may be about 40 fps and the number of pixels in each frame captured may be about 8.3 million pixels. The light detector 436 is mounted to the housing 411. When assembled, the aperture 432 lies adjacent to the CMOS sensor 438 and is configured to allow light to pass. The detection package 410 also includes an electrical connection 439 that is in electrical communication with a computing device. In other implementations, a wireless transmitter may be included and the electrical connection 439 may be omitted. The components of detection package 430 are examples of components that can be implemented in the detection package 130 of apparatus 100 in FIG. 1 and the detection packages of the multi-channel apparatus 700 in FIGS. 7A and 7B.

In various embodiments, one or more operations of a method for determining the one or more cerebral blood metrics may be implemented by one or more computing devices or one or more processors. For example, such a computing device and/or processor may be configured to analyze data from the light detector and/or generate one or more cerebral blood flow metrics. In some implementations, the one or more computing device and/or one or more processors may be disposed on the same headset as the one or more light emission packages and one or more light detector packages. Additionally or alternatively, the one or more computing devices and/or one or more processors may be communicatively coupled to the headset, e.g., by a wireless or wired communication channel.

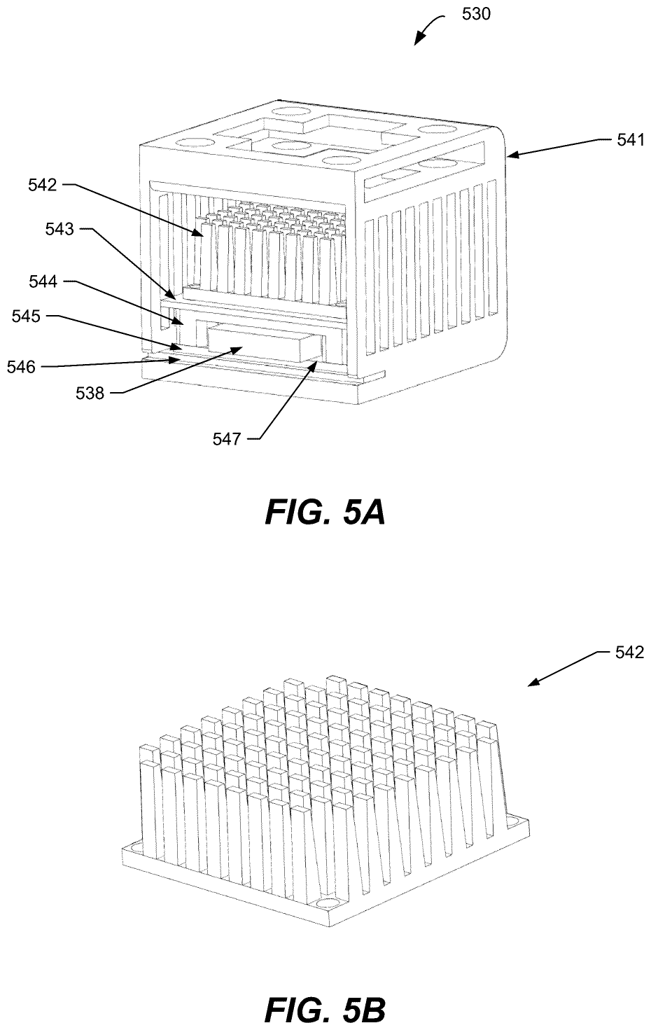

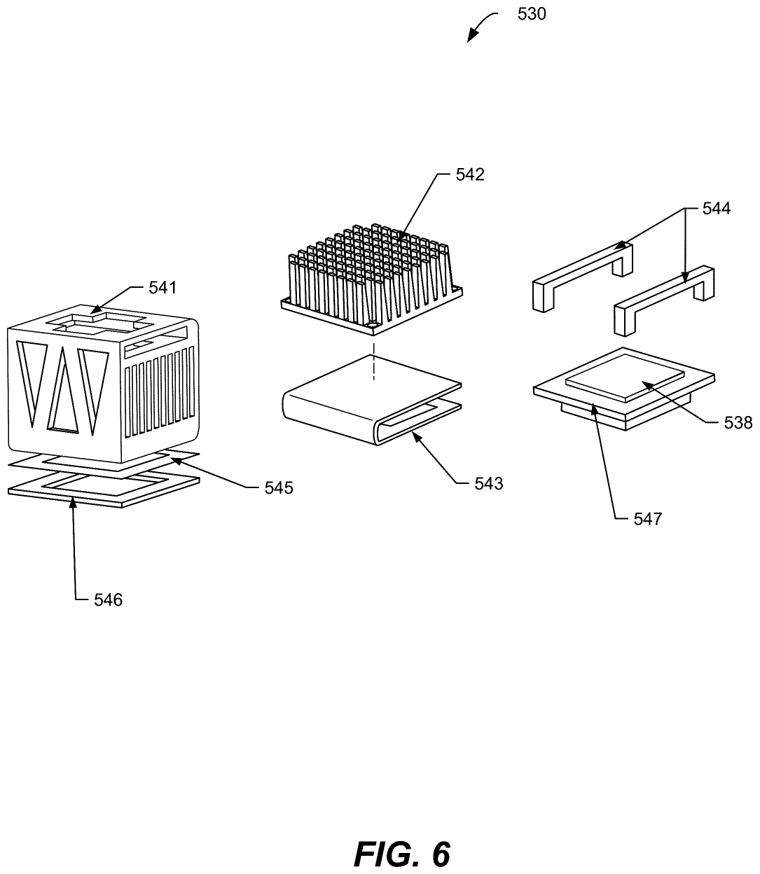

FIG. 5A is a drawing of an isometric view of a detection package 530 having a heat management system including a heat sink 542, a heat spreader 543, one or more standoffs 544, a first thermally insulative layer 545 (e.g., a cork layer such as a 1 mm layer of cork) and a second thermally insulative layer 546 (e.g., a neoprene or silicone layer), according to some embodiments. In one implementation, the second thermally insulative layer 546 is a silicone layer that allows for easy and thorough sanitization. FIG. 5B is a drawing of an isometric view of heat sink 542 in FIG. 4A. FIG. 6 is a drawing of an exploded view of the detection package 530 in FIG. 5A.

The heat spreader 543 is located adjacent to the heat sink 542. The heat spreader 543 includes a plate of thermally conductive material (e.g., copper) that is U-shaped such that the USB-board camera may be located between the two outer walls to extract heat away from the USB-board camera to the heat sink 542. The heat sink 542 is made of a thermally conductive material (e.g., an anodized aluminum) that includes a plurality of prongs or teeth for conducting heat to the ambient environment. The detection package 430 also includes a housing 541 (e.g., a 3-D printed structure) within which components of the heat management system and the light detector 436 are contained and/or mounted.

FIG. 7A depicts a schematic illustration of an isometric view of a multi-channel apparatus 700 for generating cerebral blood metrics data, according to some embodiments. FIG. 7B depicts a schematic illustration of a top view of the multi-channel apparatus 700 in FIG. 7A. The multi-channel apparatus 700 includes a headset 101 with a headband 702 encircling a head 20 of a user. The headset 101 has six channels 741, 742, 743, 744, 745, and 746. Additional or fewer channels may be used in other implementations.

Each channel includes a source-detector pair. The first channel 741 includes a first source-detector pair including a first light source 711 (e.g., a laser that can emit light in a near-infrared or infrared wavelength) and a first detector 731, the second channel 742 includes a second source-detector pair including the first light source 711 and a second detector 732, the third channel 743 includes a third source-detector pair including a second light source 712 and a third detector 733, the fourth channel 744 includes a fourth source-detector pair including the second light source 712 and a fourth detector 734, the fifth channel 745 includes a fifth source-detector pair including a third light source 713 and a fifth detector 735, and the sixth channel 746 includes a sixth source-detector pair including the third light source 713 and a sixth detector 736. The source-detector pairs of the six channels may be located along the headband 702 to probe different regions of the brain simultaneously or at different times. For example, the source-detector pairs may be located to measure cerebral blood metrics at right and left frontal lobe regions, right and left parietal lobe regions, right and left temporal lobe regions, or the like. In one implementation, the multi-channel apparatus 700 may be used to generate cerebral blood metrics data that can be used to detect traumatic brain injury. In some cases, the multi-channel apparatus 700 is configured to be able to adjust the locations of the light emission packages with the light sources and the light detector packages with light detectors along the headband 702.

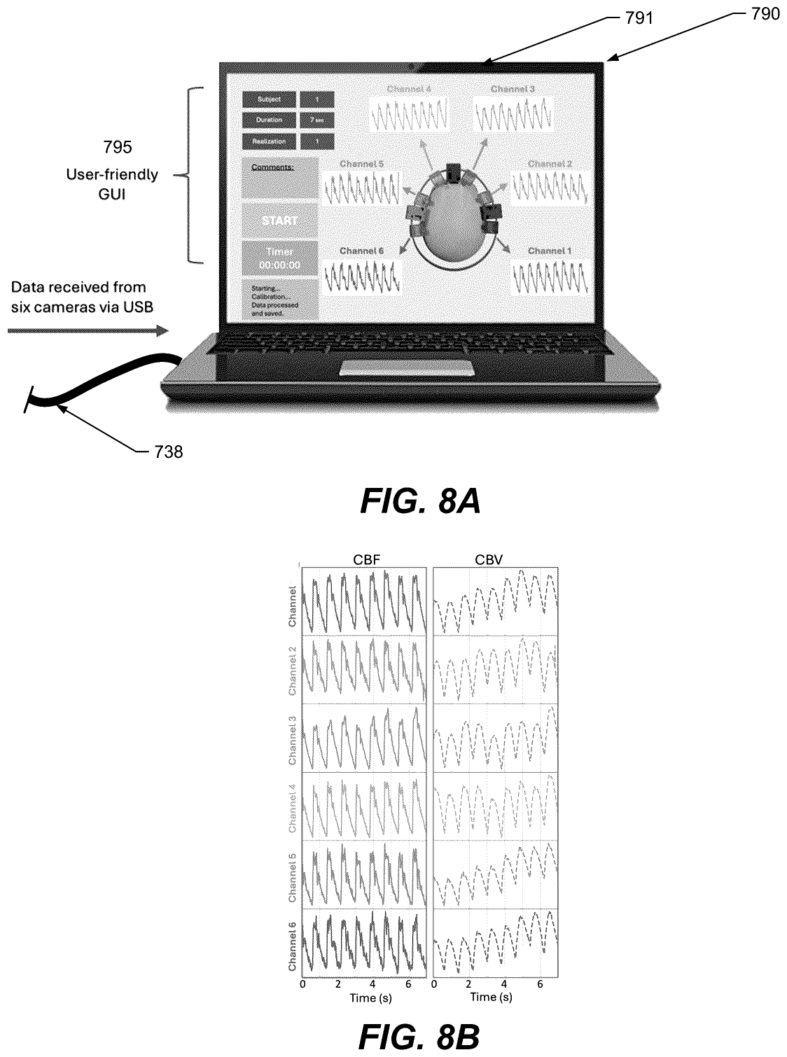

The multi-channel apparatus 700 also includes a first power source 781 (e.g., a 9 v bolt battery or the like) electrically coupled to the first light source 711, a second power source 782 electrically coupled to the second light source 712, and a third power source 783 electrically coupled to the third light source 713. In addition, the multi-channel apparatus 700 includes a computing device 790 and electrical connectors 738 between the light sources and the computing device 790 and between the light detectors and the computing device 790. In other implementations, wireless communication may be employed. The computing device 790 may be part of the multi-channel apparatus 700 or may be a separate component as shown in FIGS. 7A and 8A.

The computing device 790 includes a display 791 for providing a graphical user interface (GUI) 795 that may be used to control the multi-channel apparatus 700. The GUI 795 may also be used for visualization of the cerebral blood flow metrics. For instance, in FIG. 8A, GUI 795 is shown displaying plots of the time traces of the cerebral blood flow measured at the six regions of the brain being probed by the source-detector pairs of the six channels 741, 742, 743, 744, 745, and 746. FIG. 8B illustrates examples of plots of time traces of cerebral blood flow and cerebral blood volume measured by the six channels. In some cases, the computing device 790 may be used to perform real-time processing of the image data to extract the cerebral blood metrics which may eliminate the need for computing resources for storing the raw images.

FIG. 9A depicts a schematic illustration of an isometric view of an apparatus 900 for generating cerebral blood metrics data, according to some embodiments. FIG. 9B depicts a schematic illustration of a top view of the apparatus 900 in FIG. 9A. FIG. 9C depicts a schematic illustration of a side view of the apparatus 900 in FIG. 9A. Apparatus 900 includes a laser diode 914 and a light detector 936 mounted to a circuit board 902 (e.g., printed circuit board (PCB)) forming a source-detector package with a low profile. The source-detector package may include a housing enclosing more components of the apparatus 900 or another structure for attaching to a headset. In some cases, multiple source-detector packages may be attached to the headset.

The circuit board 901 may have a length in a range of 3 cm and 5 cm and a width in a range of 3 cm and 5 cm. In one example, the circuit board 902 has a length of 5 cm and a width of 3 cm. In one example, the thickness of the circuit board 902 is about 1 mm. In some cases, the circuit board 902 may be made of a flexible material.

The laser diode 914 may be configured to emit light in a near-infrared or infrared wavelength. In one example, the laser diode 914 may emit light at about 830 nm. In another example, the laser diode 914 may emit light at about 785 nm. In one instance, the laser diode 914 may be a single-mode continuous wave 785 nm laser diode (e.g., Thorlabs laser L785H1) that can deliver up to 200 mW. The light detector 936 includes dimensions of length, l, and width, w. In the illustrated example, the light detector 936 is elongated having a length dimension longer than the width dimension. In some cases, light detector 936 may have a length, l, in a range of 15 mm and 25 mm a width in a range of 5 mm and 10 mm.

In some implementations, the light detector 936 may be segmented into a plurality of detector segments that can simultaneously acquire images. In one implementation, the light detector 936 may be comprised of a plurality of sensors (e.g., CMOS sensors) arranged along the length, l, that can capture image frames simultaneously of different regions having different depths based on the corresponding S-D distances from the light diode 914. By way of example, the light detector 936 may be comprised of three sensors arranged along the lengthwise dimension including a first sensor closest to the laser diode 914, a second sensor, and third sensor furthest from the laser diode 914. The third sensor may acquire images at a greater depth than the images acquired by the first sensor and the second sensor. In another implementation, the light detector may include a sensor with a two-dimensional array of pixels divided into different sets (segments) of pixels that can simultaneously acquire images of different regions.

The apparatus 900 also includes a light block 950 made of a wall of opaque material located between the laser diode 914 and the light detector 936 to help prevent cross talk. Some examples suitable opaque materials include black plastic, PLA, and 3D printing black resins. In other implementations, one or both the laser diode 914 and the light detector 936 may be enclosed within an enclosure of opaque material or other light blocking structure may be used. The light block 950 may have a length in a range of 2 cm and 5 cm a height in a range of 3 mm and 1 cm. The apparatus 900 also includes a wireless transmitter 987 for sending cerebral blood metrics data such as time traces via a wireless communication connection such as BLUETOOTH or wifi to an external computing device 990 (e.g. a cell phone).

The apparatus 900 also includes an onboard computing device 980 including one or more processing units or processors (e.g., an application-specific integrated circuit (ASIC) or a programmable logic device such as a field-programmable gate array (FPGA)) coupled to the circuit board 902. The processing units or processors may be configured with instructions for performing one or more operations of the apparatus 900. For example, the one or more processing units or processors of the onboard computing device 980 may be configured to analyze data from the light detector 836, generate cerebral blood flow metric data, and/or provide data representative of the cerebral blood flow metrics such as time traces to a second computing device 990. The apparatus 900 also includes a power source 985 in electrical communication with the laser diode 914 and the one or more processing units or processors 980 for providing power. The power source 985 may be a rechargeable battery, for example.

In various embodiments, an apparatus for determining cerebral blood metrics includes one or more processing units or processors. A processor can be implemented as a microcontroller or as one or more logic devices including one or more application-specific integrated circuits (ASICs) or programmable logic devices, such as field-programmable gate arrays (FPGAs) or complex programmable logic devices. If implemented in a programmable logic device, the processor can be programmed into the programmable logic device as an intellectual property block or permanently formed in the programmable logic device as an embedded processor core. In some other implementations, a processor can be or can include a central processing unit, such as a single core or a multi-core processor. The processor is coupled to memory and/or memory may be integrated in the processor, for example, as a system-on-chip package, or in an embedded memory within a programmable logic device itself.

### Methods of Determining Blood Flow Metrics

In some implementations, cerebral blood metrics may include one or more of cerebral blood flow, cerebral blood volume, and heart rate. In some embodiments, cerebral blood volume may be determined based on the intensity of reflected light as measured by a light detector. Note that cerebral blood volume may be determined based on reflected light of a single wavelength (e.g., emitted from a laser).

In some embodiments, cerebral blood flow may be determined using collected scattered light at a single wavelength (e.g., light emitted in an infrared or near infrared wavelength range). The wavelength implemented may affect penetration depth of the light. For example, light emitted in the infrared wavelength range may penetrate to deeper structures relative to near infrared light. In some implementations, cerebral blood flow measurements may be based on speckle visibility spectroscopy (SVS), which is also referred to as speckle contrast optical spectroscopy (SCOS). In general, a “speckle” refers to the pattern of bright and dark spots in an image resulting from scattering of illuminated laser light (e.g., scattered by the scalp, skull, and/or brain) resulting from constructive and destructive interference of the light. The speckle pattern dynamics change with blood flow dynamics. The time that it takes one speckle pattern to change to a different speckle pattern is referred to as the decorrelation time, which is correlated with cerebral blood flow rate. SVS techniques measure how fast the speckle pattern changes, and hence, estimating a cerebral blood flow rate. SVS typically determines speckle decorrelation time using a relatively slow camera with a large number of pixels. In some cases, the camera exposure time, typically in the range of 0.3 milliseconds to 15 milliseconds, may be set to be substantially longer than the decorrelation time of the speckle field (typically a few tens of microseconds), which may result in multiple different speckle patterns summed up into a single camera frame. As the speckle field fluctuates, the speckle pattern recorded by the camera is smeared and washed out within the exposure time. Accordingly, the cerebral blood flow may be quantified from the degree of blurring of the captured frame, which is generally referred to herein as the speckle contrast.

In one implementation, raw images are normalized to remove nonuniform intensity distributions. A speckle contrast is calculated from the normalized images and the calculated speckle contrast is adjusted to remove the influence of camera noise, shot noise, and quantization noise. The adjusted speckle contrast can be used to determine the cerebral blood flow measured in units of a normalized blood flow index. An example of a process flow for this implementation is shown in FIG. 10.

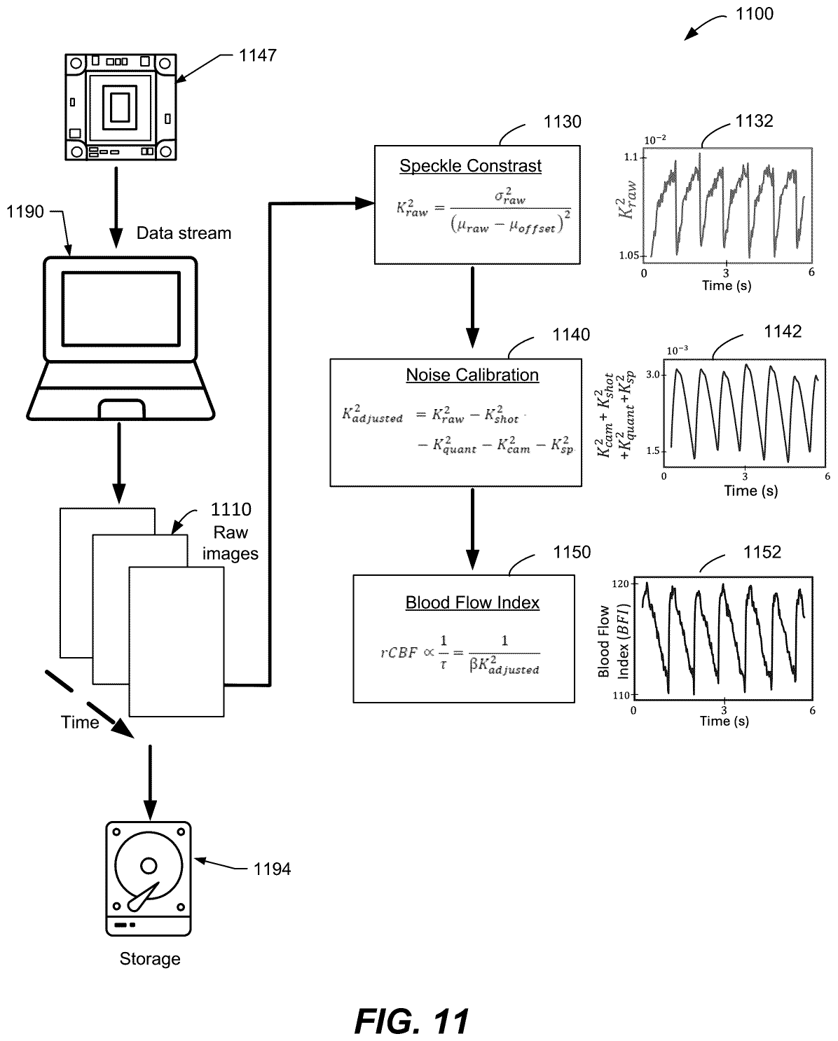

In one implementation, a raw speckle contrast is calculated directly from the raw speckle images. The raw speckle contrast is adjusted to remove the influence of camera noise, shot noise, quantization noise, and spatial inhomogeneity. The adjusted speckle contrast can be used to determine the cerebral blood flow measured in units of a blood flow index. An example of a process flow for this implementation is shown in FIG. 11.

BFI is typically a relative measurement of blood flow velocity. Blood flow is generally related to blood pressure, the radius of the blood vessel, dynamic viscosity of the blood, and the length of the blood vessel. Any alteration in blood flow means a change in either the blood pressure or diameter of the blood vessel. A slight adjustment in the radius of the blood vessel can substantially impact blood flow due to the fourth power relationship of blood flow with the radius of the blood vessel. Comparing BFI values collected at different time points can serve as an indicator of changes in blood flow velocity.

However, it's important to emphasize that BFI is a relative metric and should not be compared across different experimental conditions, including but not limited to, different S-D distances, different locations on the head or different subjects, and illumination wavelengths. This is because τ can be influenced not only by the velocity of blood flow (BFI) but also by the blood flow volume (BFV), the presence of local vascular structures, and the blood pressure (1). Therefore, the translation from K_{adjusted}² to BFI is a relative approach and is commonly denoted as rBFI or normalized BFI in the literature (24-26). Relative to the typical SVS systems (15, 16, 19),

The use of SVS to measure cerebral blood flow may have the advantage that a relatively inexpensive camera may be used as a light detector, because a high frame rate is not needed. Moreover, a camera may be directly mounted on a headband, which may eliminate optical loss associated with a light guide running from the head to an external camera, which may introduce its own noise and motion artifacts. In some examples, a camera may have an integration exposure time of within a range of about 0.3 milliseconds-15 milliseconds. The camera may use a frame rate of within a range of about 30 frames per second-150 frames per second. In one example, the camera may have a frame rate of about 80 frames per second. In one example, the camera may have a frame rate of about 50 frames per second. Moreover, optical fibers are generally limited to 600 or 1000-microns in diameter whereas CMOS sensor sizes can be a square of dimension ranging from 1 mm to 10 mm, which is significantly larger. Positioning CMOS sensors directly atop the region of interest not only provides a stable placement but also provides a larger collection area and numerical aperture for collecting photons. Also, sensors may be divided into segments that allow a single light detector to capture multiple images at different S-D distances. For example, the light detector 836 in FIGS. 9A-C may be divided into three segments.

In addition to monitoring cerebral blood flow, apparatus described herein may also analyze intensity changes in the images captured by the light detector to take absorption measurements that determine changes in cerebral blood volume (CBV). In some cases, the light detector is configured to emit an illumination wavelength of 785 nm, which may minimize the influence of oxygen concentration changes.

### Example 1

FIG. 10 depicts an example of a process flow for determining cerebral blood flow from raw images 1010, according to embodiments. Components of an apparatus for determining blood flow metrics (e.g., apparatus 100 in FIG. 1, multi-channel apparatus 700 in FIG. 7, apparatus 800 in FIG. 8) may be used to implement the process flow. The cerebral blood flow may be measured in units of blood flow index (BFI). The cerebral blood volume may be measured in units of blood flow index (BVI). As shown, a light detector 1047 (e.g., a camera) of the apparatus captures a plurality of raw images 1010. The exposure time of the light detector 1047 is substantially longer than the decorrelation time of the speckle field such that multiple speckle patterns are integrated into each raw speckle image. The raw images 1010 may be stored to and retrieved from a non-transitory computer readable medium 1094.

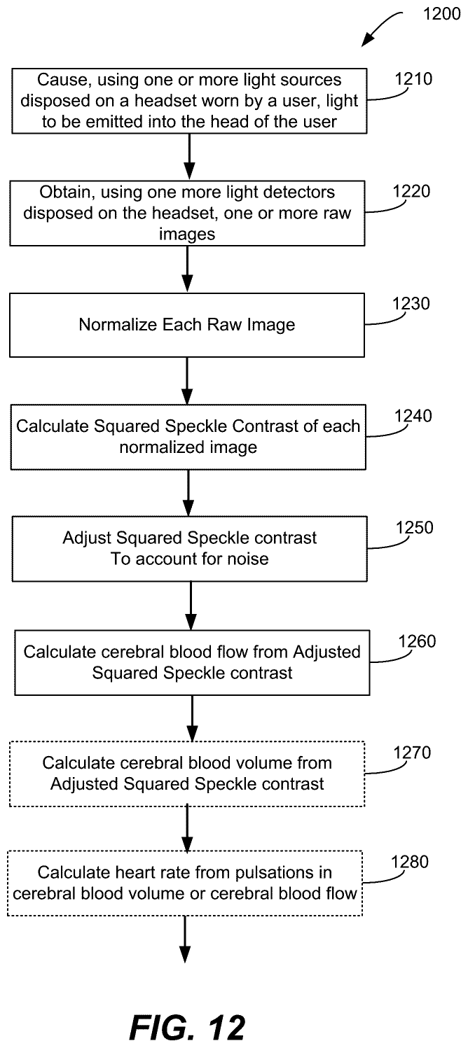

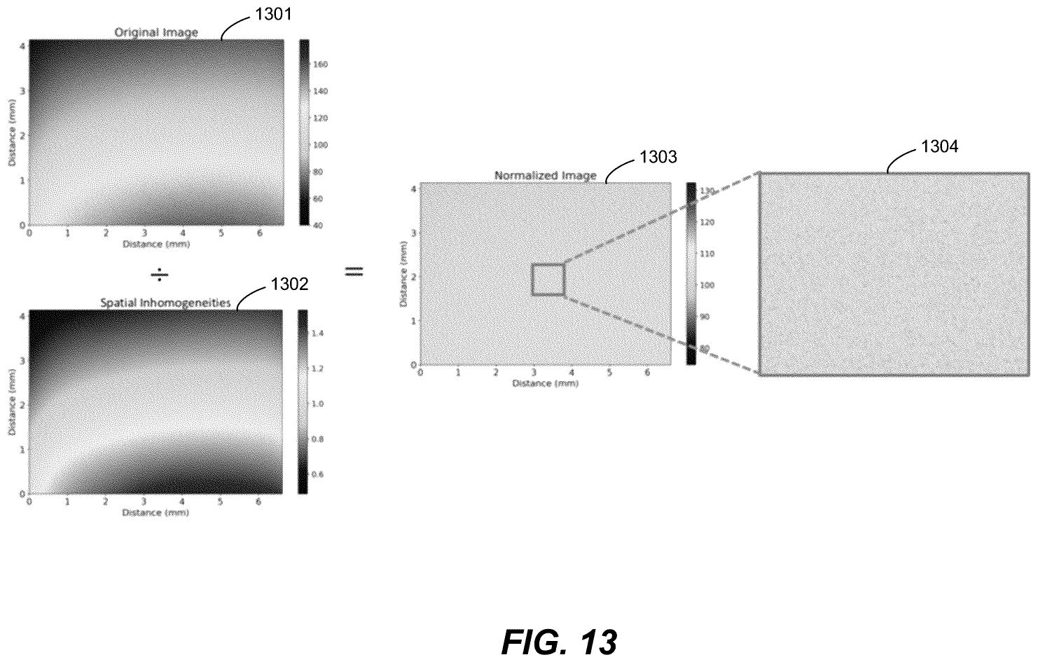

The raw images may have nonuniform intensity distributions. For example, since one side of the light detector is closer to the light source, the pixels closer to the light source will have higher intensities than those further away. To remove the nonuniform intensity distribution from the images, each raw image is normalized (block 1020) by dividing each raw image by a mean image. The mean image is determined from a plurality of raw images acquired in a calibration operation, typically taking place in a time period before the plurality of raw images 1010 are acquired. An example of a normalization operation is described in more detail with reference to FIG. 12.

At block 1030, the squared speckle contrast may be determined at each normalized image, I, as:

$${K}_{raw}^{2} ( I ) = \frac{{σ}^{2} ( I )}{{μ}^{2} ( I )} \qquad ( Eqn . 1 )$$

In Eqn. 1, σ²(I) represents the variance of the normalized image I, and μ represents the mean of the normalized image I. An example of the squared speckle contrast as a function of time is shown in graph 1032.

At block 1040, the adjusted squared speckle contrast of each normalized image, I, that accounts for noise may be determined by:

$${K}_{adjusted}^{2} ( I ) = {K}_{raw}^{2} ( I ) - {K}_{shot}^{2} ( I ) - {K}_{quant}^{2} ( I ) - {K}_{cam}^{2} ( I ) \qquad ( Eqn . 2 )$$

In Eqn. 2, K²ₛₕₒₜ(I) is the contribution to variance of the images from shot noise, K²_{quant} (I) is the contribution to variance of the images from quantization, and K²_{cam}(I) is the contribution to variance of the images from camera readout noise. An example of the contributions to variance from shot noise, camera noise, and quantization noise as a function of time is shown in graph 1042.

The contribution to variance of the images from shot noise, K²ₛₕₒₜ(I) may be determined for each normalized image as:

$${K}_{shot}^{2} ( I ) = \frac{γ}{μ ( I )} \qquad ( Eqn . 3 )$$

In Eqn. 3, γ is the analog to digital conversion ratio associated with the light detector 1047, which depends on the gain setting and the conversion factor (CF), and can be determined by:

$$γ = \frac{gain}{CF} \qquad ( Eqn . 4 )$$

The contribution to variance of the images from quantization, K²_{quant}(I) may be determined for each normalized image as:

$${K}_{quant}^{2} ( I ) = \frac{1}{12 {μ ( I )}^{2}} \qquad ( Eqn . 5 )$$

The contribution to variance of the images from camera noise, K²_{cam}(I) may be determined for each normalized image as:

$${K}_{cam}^{2} ( I ) = \frac{{σ}_{cam}^{2}}{{μ ( I )}^{2}} \qquad ( Eqn . 6 )$$

In Eqn. 6, σ²_{cam} is the camera noise, which can be estimated by calculating the variance of a plurality of raw images (e.g., 100, 200, 300, 400, 500, 600, etc.) acquired in the absence of light being emitted by the light source.

At block 1050, the blood flow index at the time of image acquisition, t, can be determined by:

$$BFI ( t ) = \frac{1}{{K}_{adjusted}^{2} ( I )} \qquad ( Eqn . 7 )$$

An example of the blood flow index as a function of time is shown in graph 1052.

Blood volume measurements are based on the light absorbed by the tissues being probed and can be determined from intensities in image frames. The cerebral blood volume may be measured in units of cerebral blood volume index (BVI). The cerebral blood volume can be determined at each raw image acquisition time, t, as:

$$BVI ( t ) = \frac{2 {I}_{0} - 〈 I ( t ) 〉}{{I}_{0}} \qquad ( Eqn . 8 )$$

In Eqn. 8, I(t) is the mean pixel intensity value of the raw image and I₀ is the mean pixel intensity value of a baseline image taken during a baseline time period before the breath-holding time period.

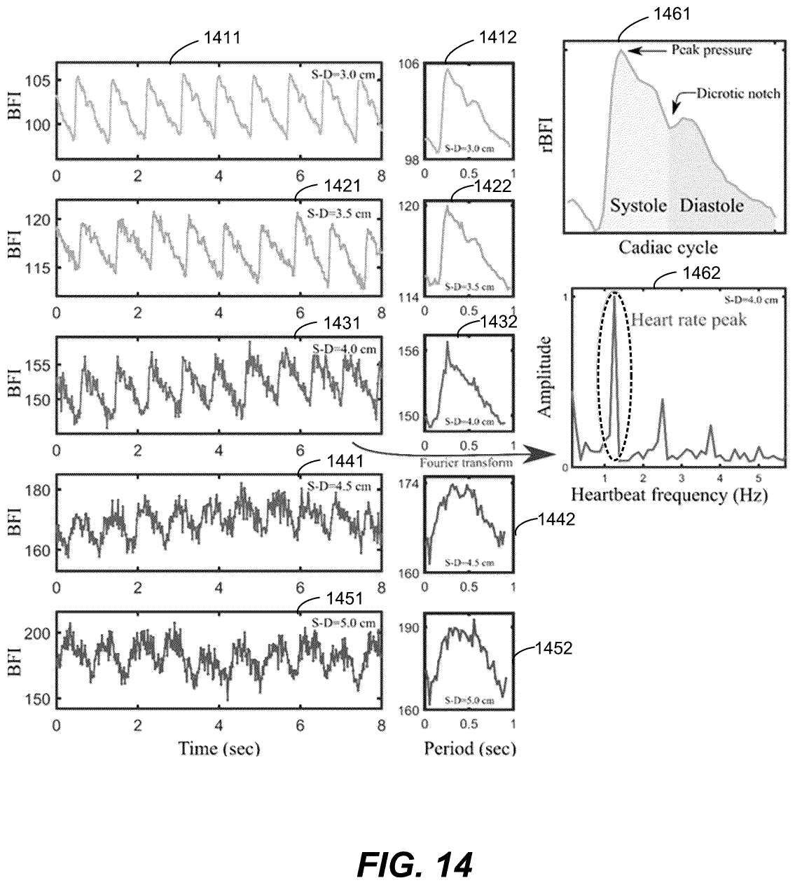

The heart rate can be determined from the pulsations in the blood flow or blood volume measurements over time, which is described in more detail below with reference to FIG. 11 and FIG. 14. For example, the heart rate may be determined by taking a Fourier transform of time domain data such as time traces of cerebral blood flow or cerebral blood volume. The heart rate can be determined as the first sub-harmonic peak of the pulsations in the frequency domain.

### Example 2

FIG. 11 depicts another example of a process flow for determining cerebral blood flow, according to embodiments. Components of an apparatus for determining blood flow metrics (e.g., apparatus 100 in FIG. 1, multi-channel apparatus 700 in FIG. 7, apparatus 800 in FIG. 8) may be used to implement the process flow. As shown, a light detector 1147 (e.g., camera) of the apparatus captures a plurality of raw speckle images 1110. The exposure time of the light detector 1047 is substantially longer than the decorrelation time of the speckle field such that multiple speckle patterns are integrated into each raw speckle image. The images 1110 may be stored to and retrieved from a non-transitory computer readable medium 1194.

At block 1130, to quantify the fluctuations of the speckle field, the raw squared speckle contrast may be determined at each raw image as:

$${K}_{raw}^{2} = \frac{{σ}_{raw}^{2}}{{( {μ}_{raw} - {μ}_{offset} )}^{2}} , \qquad ( Eqn . 8 )$$

In Eqn. 8, σ_{raw} is the standard deviation and μ_{raw} is the mean of the recorded image, and μ_{offset} accounts for the light detector offset. To experimentally measure the offset, a series of images are captured without any source illumination, and the mean value μ_{offset} is calculated of all the captured images. An example of the raw squared speckle contrast as a function of time is shown in graph 1132.

At block 1140, the adjusted raw squared speckle contrast of each raw speckle image that accounts for noise may be determined by:

$${K}_{adjusted}^{2} = {K}_{raw}^{2} - {K}_{shot}^{2} - {K}_{quant}^{2} - {K}_{cam}^{2} - {K}_{sp}^{2} , \qquad ( Eqn . 9 )$$

In Eqn. 9, K²ₛₕₒₜ is the contribution to variance of the images from shot noise, K²_{quant} is the contribution to variance of the images from quantization, K²_{cam} is the contribution to variance of the images from camera readout noise, and K²ₛₚ is the contribution to variance of the images from spatial inhomogeneities. An example of the contributions to variance from this noise as a function of time is shown in graph 1142.

The contribution to variance of the images from shot noise, K²ₛₕₒₜ may be determined for each raw image as:

$${K}_{shot}^{2} = ( \frac{γ}{{μ}_{raw} - {μ}_{offset}} ) , \qquad ( Eqn . 10 )$$

In Eqn. 10, the γ is the analog to digital conversion ratio associated with the light detector, which depends on the gain setting and CF of the light detector and can be determined by Eqn. 4. In some examples, the gain is set in a range of 1 to 72, corresponding to a 0 to 37 dB setting. In one example, the light detector 1047 is a Basler board camera with a conversion factor of CF=40.7.

The contribution to variance of the images from quantization, K²_{quant} may be determined for each raw image as:

$${K}_{quant}^{2} = ( \frac{1}{12 {( {μ}_{raw} - {μ}_{offset} )}^{2}} ) , \qquad ( Eqn . 11 )$$

The contribution to variance of the images from camera noise, K²_{cam} may be determined for each raw image as:

$${K}_{cam}^{2} = ( \frac{{σ}_{cam}^{2}}{{( {μ}_{raw} - {μ}_{offset} )}^{2}} ) , \qquad ( Eqn . 12 )$$

In Eqn. 12, σ²_{cam} is the camera noise, which can be estimated by calculating the variance of a plurality of raw images (e.g., 100, 200, 300, 400, 500, 600, etc.) acquired in the absence of light being emitted by the light source.

The contribution to variance of the images from spatial inhomogeneities, K²ₛₚ may be determined for each raw image as:

$${K}_{sp}^{2} = ( \frac{{σ}_{sp}^{2}}{{( {μ}_{raw} - {μ}_{offset} )}^{2}} ) . \qquad ( Eqn . 13 )$$

In Eqn. 13, σ²ₛₚ is the noise from spatial variations in photon flux across the light detector area.

At block 1050, the relative cerebral flow can be determined as:

$$rCBF ∝ \frac{1}{τ} = \frac{1}{β {K}_{adjusted}^{2}} \qquad ( Eqn . 14 )$$

The speckle decorrelation time is directly correlated with the cerebral blood flow rate. The relative cerebral blood flow, measured in units of blood flow index (BFI), is inversely correlated with τ and therefore inversely correlated with βK². The correction factor β is a constant depending on system setup, e.g. speckle size, pixel size, polarization of the laser light.

The relative cerebral blood volume can be determined for each raw image as:

$$rCBV = \frac{1}{{μ}_{raw} - {μ}_{offset}} . \qquad ( Eqn . 15 )$$

### Example 3

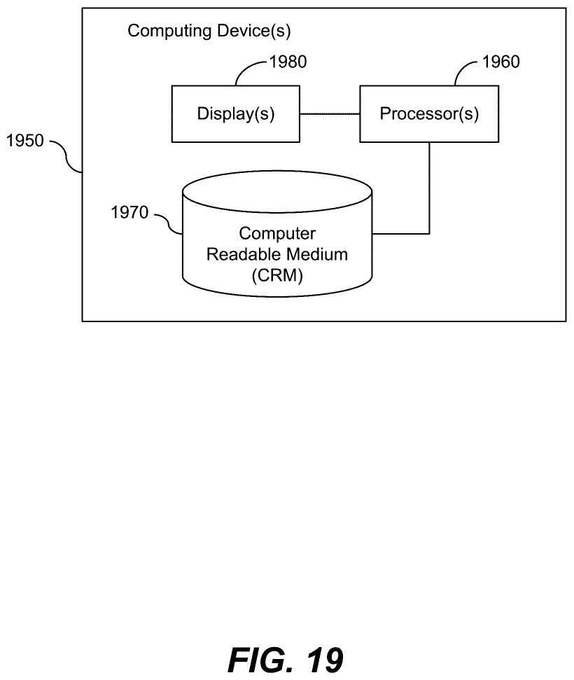

FIG. 12 is a flowchart of an example process for determining cerebral blood metrics in accordance with some embodiments. Blocks of process 1200 may be executed by one or more processors of one or more computing devices. An example of such a computing device is shown in and described below in connection with FIG. 19. Note that, in some embodiments, at least one of the one or more computing devices may be disposed on the headset on which the one or more light sources and light detectors are disposed. For example, the apparatus 800 with computing device 880 may be attached to a headset. Accordingly, in some such embodiments, cerebral blood metrics may be determined by a computing device itself on the headband or headset. Alternatively, in some embodiments, data obtained by the one or more light detectors, and/or data representative of the cerebral blood metrics may be transmitted from a computing device disposed on the headband or headset to a second computing device remote from or separate from the headband or headset, where the second computing device generates cerebral blood metrics. In some implementations, blocks of process 1200 may be executed in an order other than what is shown in FIG. 12. In some embodiments, one or more blocks of process 1200 may be omitted, and/or two or more blocks may be executed substantially in parallel.

At 1210, process 1200 can begin by causing, using one or more light sources disposed on a headset worn by a user, light to be emitted into the head of the user. Examples of such a headset are shown in and described above in connection with FIGS. 1, 2, and 7. The one or more light sources may include one or more lasers, each of which may emit light in the near infrared or infrared wavelength. In some implementations, multiple light sources are disposed on a headset or headband, each configured to emit light into different regions of the user's head or brain. The light may be emitted continuously, or may be pulsed.

At 1210, process 1200 may obtain, using one or more light detectors disposed on the headset, information indicative of light reflected from one or more structures within the head or brain of the user. The obtained information includes image data for a plurality of raw images. Each raw image is captured over an exposure time that may be substantially longer than the decorrelation time of the speckle field such that multiple speckle patterns are integrated into each raw image. An example of one or more light detectors disposed on a headset is shown in and described above in connection with FIGS. 1, 2, and 7. As described above, the one or more light detectors may include one or more CMOS sensors, a camera, etc. In some cases, a portion of the obtained information spans a time period during which the user was holding their breath.

In one example, light emitted by a laser may be reflected from various head and brain structures and may be captured by a camera (e.g., to determine speckle contrast, as described above). Note that, as shown in and described above in connection with FIG. 7, because a headset may include multiple (e.g., two, four, eight, ten, etc.) light emission packages and light detection packages, the obtained information may correspond to different brain regions (e.g., a left frontal lobe region, a right frontal lobe region, a left parietal lobe region, a right parietal lobe region, etc.).

Note that, the obtained light reflection data may span a time period that includes a baseline time period, a breath holding time period, and a recovery time period. In some implementations, process 1200 may cue the user to begin holding their breath at a particular time. For example, the cue may be an audible cue (e.g., a spoken instruction, an audible beep, etc.), or may be a haptic cue (e.g., a vibration delivered using the headset).

At 1220, process 1200 may, normalize each raw image by dividing each raw image by a mean image. The mean image refers to an averaged intensity distribution of a plurality of images obtained during a time period before the time period used to acquire the plurality of speckle images. For example, the plurality of raw images may be obtained in a time period during which the user was holding their breath (breath hold period). The mean image is determined from another plurality of images (e.g., 50, 100, 200, 300, 400, 500, etc.) obtained during a baseline period prior to the breath hold period during.

At 1230, process 1200 calculates the speckle contrast of each normalized image using Eqn. 1. At 1240, process 1200 adjust the speckle contrast to account for noise and other factors. For example, the speckle contrast may be adjusted to remove variance due to shot noise, camera noise, and quantization as provided in Eqn. 2. Contribution to variance of the images from shot noise may be determined for each normalized image by Eqn. 3. Contribution to variance of the images from quantization may be determined for each normalized image by Eqn. 5. Contribution to variance of the images from camera noise may be determined for each normalized image by Eqn. 6.

At 1250, process 1200 determines the cerebral blood flow from the adjusted speckle contrast. For example, the process 1200 may determine the cerebral blood flow in units of blood flow index using Eqn. 7.

Blood volume measurements are based on the light absorbed by the blood and tissues being probed and can be determined from intensities in image frames. Assuming the tissues will not change, the only changes in absorption are due to increasing or decreasing blood, which can be inferred as relative changes in blood volume. At optional (denoted by dashed line) 1260, process 1200 may determine the cerebral blood volume at each image acquisition time using Eqn. 8.

At optional (denoted by dashed line) 1270, process 1200 may determine the heart rate based on the periodicity of pulsations in time domain data of cerebral blood volume or the cerebral blood flow measurements. For example, the graphs 1411, 1421, 1431, 1441, and 1451 in FIG. 4 are time traces of cerebral blood flow measurements that have blood pulsations. The process 1200 can take a Fourier transform of the traces and determine the first harmonic peak in the frequency domain as the heart rate.

### Experimental Data

### S-D Distances

The distances between a light source and a light detector (S-D distances) impact the depth emitted light can penetrate within the brain and the corresponding depths of imaging and signal sensitivity. The scalp and skin of the user as well as other conditions may also affect signal sensitivity. In certain implementations, the S-D distance for a particular subject may be tuned for signal sensitivity. In some cases, an S-D distance of between 3 cm and 4.5 cm may provide an adequate signal sensitivity to detect cerebral blood flow.

The apparatus 100 in FIG. 1 was used to obtain cerebral blood metrics data from ten subjects. The headset 101 was placed on the forehead of the subjects as shown in FIG. 1. Measurements were collected over 8 seconds for each of five different source-to-detector (S-D) distances (±2 mm): 3.0 cm, 3.5 cm, 4.0 cm, 4.5 cm, and 5.0 cm for each subject.

FIG. 14 illustrates examples of experimental cerebral blood metric data collected over 8 seconds for five different source-to-detector (S-D) distances (±2 mm): 3.0 cm, 3.5 cm, 4.0 cm, 4.5 cm, and 5.0 cm for a first subject. As illustrated by graphs 1411, 1421, 1431, 1441, and 1451, cerebral blood flow is monitored in units of blood flow index during an 8 second time period by a light detector at source-to-detector (S-D) distances of 3.0 cm, 3.5 cm, 4.0 cm, 4.5 cm, and 5.0 cm. In this example data for the first subject, an S-D distance of 3 cm provided adequate brain signal sensitivity to detect cerebral blood flow. As the S-D distance extended beyond 3.0 cm, the gain in sensitivity becomes progressively smaller, peaking at S-D distances of approximately 4 cm.

Plots 1412, 1422, 1432, 1442, and 1452 illustrate the average cardiac cycle of the blood flow index measurements in graphs 1411, 1421, 1431, 1441, and 1451. To determine the average cardiac cycle in plot 1412, the BFI signal in 1411 was partitioned into windows of duration of 1/HR seconds (HR being the heartrate frequency from graph 1462) and subsequently calculating the mean value across all these windows. Graph 1461 illustrates two peaks, labeled peak pressure and dicrotic notch, within the plot 1412 of the average cardiac cycle of the blood flow index measurements in graphs 1411. In general, peak pressure corresponds to the rapid ejection of blood during systole and the dicrotic notch is at the beginning of diastole.

The pulsations evident in each of graphs 1411, 1421, 1431, 1441, and 1451 represent blood pulsations. These pulsations may be used to determine a heart rate of the subject (e.g., based on the periodicity of the pulsations). For example, the heart rate may be determined by taking a Fourier transform of time domain data. For instance, the frequency domain graph 1462 of heartbeat frequency is a Fourier transform of the time domain data in graph 1431 of cerebral blood flow index measurements. The heart rate may be determined based on the heartbeat peak frequency (first sub-harmonic) 1463 in the frequency domain graph 1462. In the illustrated example, the heartbeat of the subject was measured at heart rate (HR)=1.25 Hz (75 bpm).

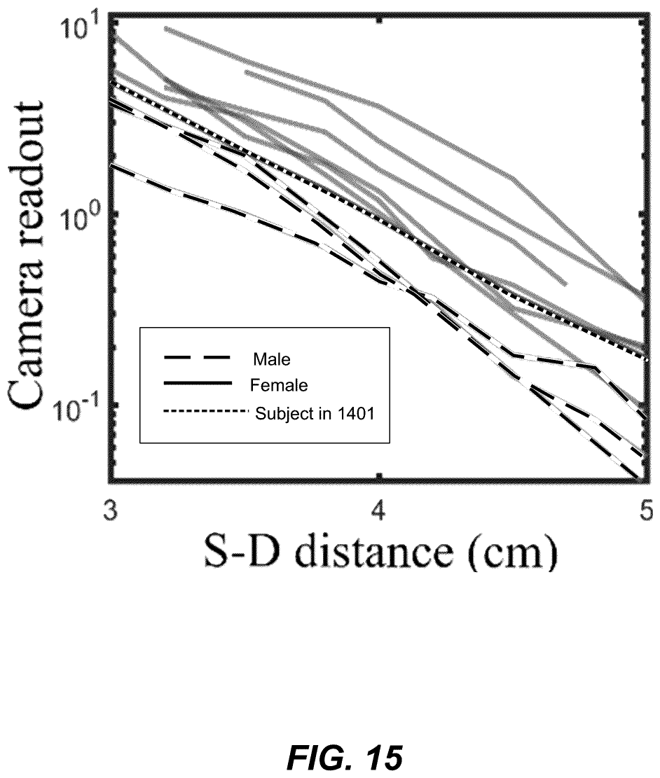

The headset 101 from FIG. 1 was then used to obtain cerebral blood metric data for an additional nine subjects. The measurements were taken during an 8 second time period by a light detector at source-to-detector (S-D) distances of 3.0 cm, 3.5 cm, 4.0 cm, 4.5 cm, and 5.0 cm. FIG. 15 depicts a plot 1501 of the average measured camera intensity as a function of the S-D distances for the ten subjects. An average measured camera intensity at each S-D distance can be calculated by taking the average intensity of all the raw images taken at that distance. The data shows that for S-D distances up to 4.5 cm for all subjects, the readout intensities were adequate (above noise level) to obtain blood metric data.

### Breath Holding for Cerebrovascular Assessment

The brain is highly complex and plays a significant role in overseeing vital bodily functions such as the rate of oxygenation and blood circulation. Those are controlled by the brain by modulating the depth and pace of breathing and by regulating blood pressure by fine-tuning both heart rate and blood vessel diameter. The brain regulates its own blood supply, ensuring a consistent delivery of oxygen and nutrients. Together, these mechanisms guarantee the maintenance of physiological conditions.

The apparatus for determining cerebral blood metrics of certain implementations can be used to assess cerebrovascular reactivity, for example, by measuring the ability of the brain to adjust cerebral blood flow in response to changing oxygen supply in the body. In some examples, the apparatus can monitor temporal cerebral blood flow changes during controlled breath modulation tasks to be able to evaluate dynamic cerebrovascular responses elicited by oxygen deprivation scenarios.

During a breath holding time period, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels. At the beginning of a breath hold, the cerebral blood flow swiftly increases although with some fluctuation. As the breath hold prolongs, cerebrovascular reactivity should exhibit escalated demand for increased blood flow to facilitate oxygen transport. When the subject concludes the breath holding phase and resumes normal respiration, the surplus oxygen in the brain is released, and cerebral activity returns to its previous stable state.

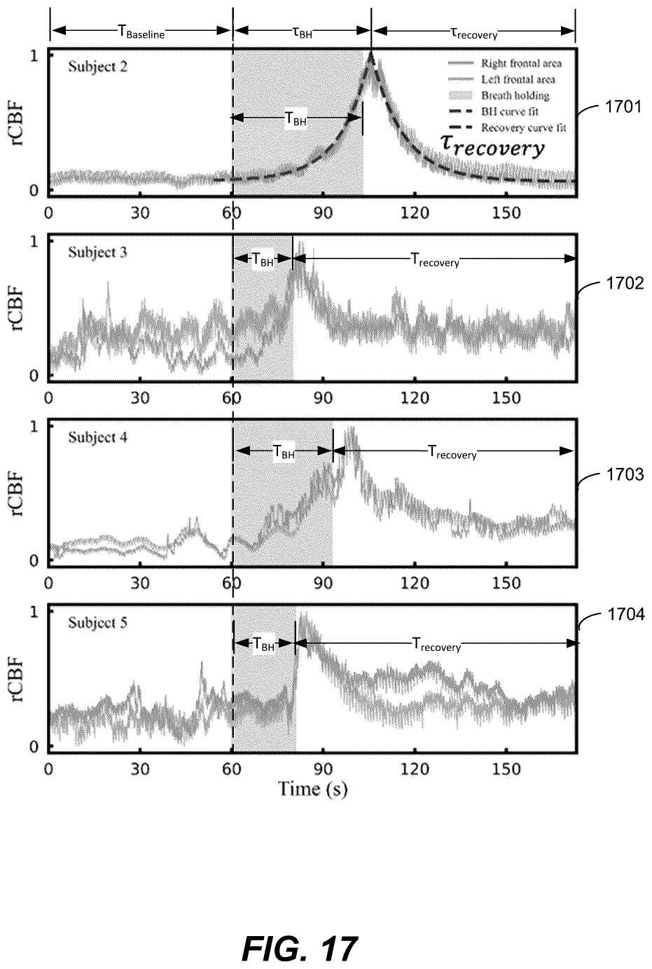

Cerebral blood metrics may be obtained over a breath holding time period during which the patient is holding their breath. Examples of breath holding time periods, T_{BH}, are illustrated in FIG. 17. Cerebral blood metrics may also be obtained over a baseline time period, generally referring to the time period during which cerebral blood metrics are obtained prior to initiation of breath holding. Examples of baseline time periods, TBaseline, are illustrated in FIG. 17. Cerebral blood metrics may also be obtained during a recovery time period, which generally begins at a time from when breath holding ends (e.g., the end of breath holding time period). Examples of recore4y time periods, Trecovery, are illustrated in FIG. 17. Note that each time period may be any suitable duration of time. Example duration of breath holding may be 15 seconds, 30 seconds, 45 seconds, 60 seconds, etc. In some embodiments, the duration of breath holding may be however as long a patient can hold their breath relatively comfortably (e.g., the breath holding time period may vary for different people). In some implementations, the baseline time period and/or the recovery time period may be at least as long as the breath holding time period. In some embodiments, the recovery time period may be longer than the breath holding time period. In some embodiments, the recovery time period may be a time duration long enough that cerebral blood flow metrics return to within a predetermined range of the corresponding values during the baseline time period. In some such embodiments, the recovery time period may be dynamically adjusted. For example, the recovery time period may be stopped responsive to determined that cerebral blood metrics have returned to baseline values.

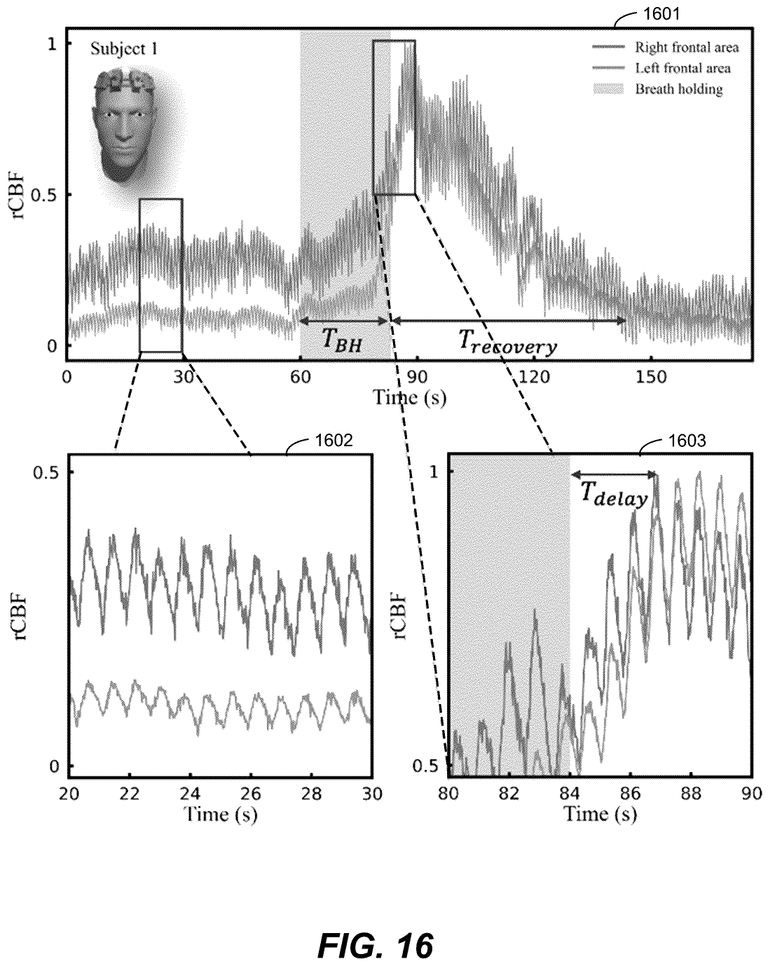

As depicted in an inset in the illustration 1601 in FIG. 16, a multi-channel apparatus 1620 with two channels was used to collect cerebral blood flow data during a breath holding task in accordance with some embodiments. Measurements of cerebral blood flow were taken at two distinct locations on the subject's forehead as illustrated.

FIG. 16 illustrates examples of experimental relative cerebral blood flow data collected during a breath holding task in accordance with some embodiments. As illustrated by a relative cerebral blood flow graph 1601, cerebral blood flow is determined during a time period that includes baseline time period 1610 during which the subject is breathing normally, a breath holding time period 1611 during which time the patient is holding their breath, and a recovery time period 1612 after the patient resumes normal breathing, and a post-recovery time period 1613 after the recovery time period. For the breath holding task, a subject is asked to breathe normally for 60 seconds. At the 60th second, the subject was prompted to fully exhale and then voluntarily hold their breath for as long as deemed comfortable. Graph 1602 illustrates a subset of the relative cerebral blood flow graph 1601 over a short time scale during the baseline time period. Graph 1603 illustrates a subset of the relative cerebral blood flow graph 1601 over a short time scale during the breath hold time period. The high frequency fluctuations in the time trace of the relative cerebral blood flow data are cardiac blood pulsations, not noise.

As illustrated in graphs 1601 and 1603, the relative cerebral blood flow increases during breath holding time period 1611. This is attributed to the brain's increased demand for blood to transport oxygen and carbon dioxide until breath holding stops. During the breath holding time period 1611, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels.

As the duration of breath hold prolongs, the brain exhibits an escalated demand for increased blood flow to facilitate oxygen transport, until breath hold task stops. Additionally, note that two to ten seconds after termination of the breath hold, the cerebrovascular reactivity still exhibits a heightened reaction, and the relative cerebral blood flow continues to increase at the beginning of the recovery time period 1612. After normal breathing resumes, the cerebral blood flow level returns to its previous baseline state.

FIG. 17 illustrates examples of experimental relative cerebral blood flow data collected during a breath holding task for four subjects, in accordance with some embodiments. As illustrated by relative cerebral blood flow graphs 1701, 1702, 1703, and 1704, cerebral blood flow is determined during a time period that includes a baseline time period (T_{Baseline}) during which the subject is breathing normally, a breath holding time period (T_{BH}) during which time the patient is holding their breath, and a recovery time period after the patient resumes normal breathing.

Various cerebral blood metrics data obtained during a breath hold task may be used as indicators of cerebrovascular health. One example metric is a duration of time a subject was able to hold their breath (generally referred to herein as T_{BH}). The range of T_{BH} can vary significantly between subjects due to differences in physical conditions and tolerance levels. An example of a T_{BH} is shown in graph 1601. Another example metric is a duration of time required for the cerebral blood flow to return to pre breath holding baseline, referred to as T_{recovery}. An example of a T_{recovery} is shown in graph 1601. Two other example metrices are τ_{recovery} and τ_{BH}, which are measured by fitting the breath holding cerebral blood flow data with an exponential curve

$$( C B F = a ⋅ exp ( \pm \frac{c}{τ} ) + c )$$

as illustrated in the graph 1701 shown in FIG. 17. Another example metric is the duration of time between the end of the breath holding period and the peak of the cerebral blood flow trace, referred to as T_{delay}. An example of a T_{delay} is shown in graph 1603. T_{delay} may serve as an indicator of the brain's response rate to abrupt changes in the body's oxygen supply. Other metrics may include a percentage change in a cerebral blood metric at a maximum or minimum after initiation of breath holding compared to a baseline value. For example, the percentage change in cerebral blood volume (CBV change) and the percentage change in cerebral blood flow (CBF change). Note that to determine a percentage change, each cerebral blood metric may be normalized based on the baseline value. Other metrics include a rate of change (e.g., a slope) in the change in a cerebral blood metric during breath holding. For example, a rate of change of cerebral blood flow may be determined by dividing a percentage change of cerebral blood flow by the duration of time the subject holds their breath, to derive a feature with units of percent change per second. Similarly, a metric may include a rate of recovery (e.g., a slope) in the change in a cerebral blood metric after resuming normal breathing. Note that, in some implementations, rates of change either during the breath holding time period, or a rate of change associated with recovery, may be determined by fitting a function (e.g., an exponential function) to a portion of the cerebral blood metric, and estimating rate of change metrics based on a growth or decay constants from the fitted function.

In some implementations, metrics may include a ratio of a percentage change of one cerebral blood metric to a percentage change of another cerebral blood metric. In one example, an metric may be a ratio of the percentage change of cerebral blood flow to the percentage change of cerebral blood volume. In some implementations, the metric may include a ratio of rate of change of one cerebral blood metric to a rate of change of another cerebral blood metric. An example includes a rate of change of cerebral blood flow to a rate of change of cerebral blood volume. Note that ratios of rates of change may be determined based on rate of change either during the breath holding time period, or during a recovery time period after normal breathing resumes.

### Cerebral Blood Volume and Heart Rate

In addition to measuring cerebral blood flow, the various apparatus described herein (sometimes referred to as SVS device or SVS apparatus) can also measure cerebral blood volume and heart rate. The heart rate can be determined based on pulsations in traces of cerebral blood flow or cerebral blood volume as discussed above with reference to FIG. 14. Cerebral blood volume measurements are based on the light absorbed by the brain and can be determined from intensities in image frames. The cerebral blood volume may be determined using Eqn. 8 or Eqn. 15.

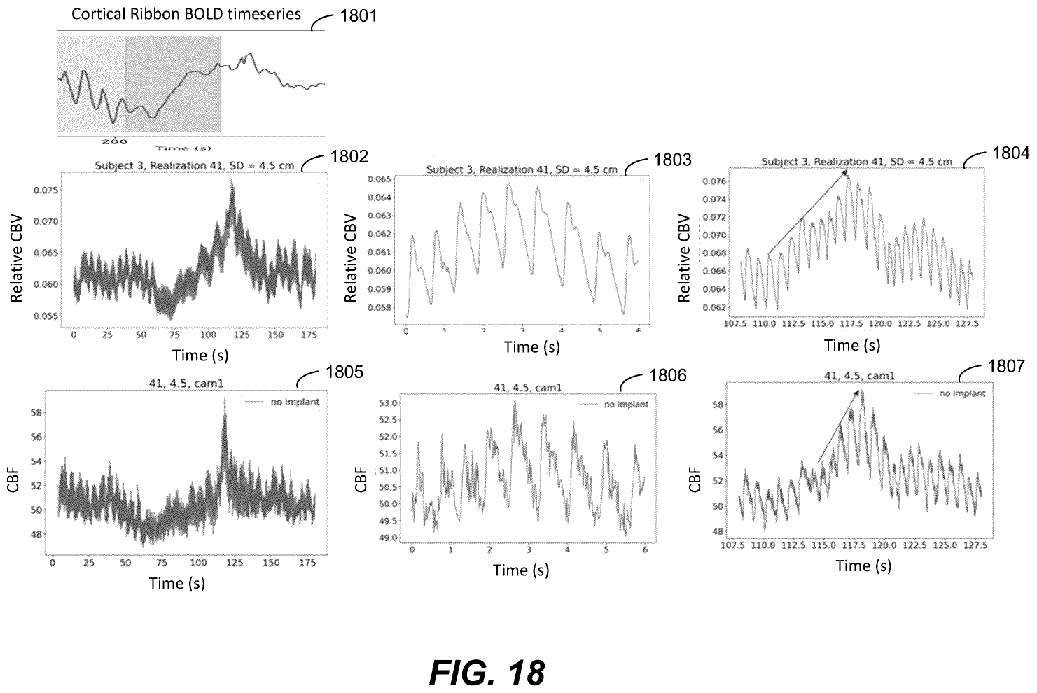

FIG. 18 illustrates examples of graphs of cerebral blood flow and cerebral blood volume in accordance with some embodiments and MRI data for comparison. In MRI experiments, subjects are asked to hold their breath for 20 seconds, followed by a 1-minute recovery window. The MRI acquired BOLD signal result is shown in graph 1801. In correspondence, the SVS device was placed on a subject with one channel on the left portion of the forehead. In the SVS experiment, the subject was asked to hold breath voluntarily starting from 60-second mark, with the total acquisition duration set at 3 minutes. In this case, the subject held their breath from 60 to 115 seconds. The breath-holding task was repeated five times, with results from one of the instances displayed in graphs 1802-1807 shown in FIG. 18. Both CBV (graphs 1802-1804) and CBF (graphs 1805-1807) show a similar trend in comparison to the MRI acquired BOLD signal. When zoomed in, the blood flow dynamics within a cardiac period can be observed, e.g. graph 1806. CBF traces will demonstrate additional higher frequency details in comparison to CBV. Based on overall trend, the CBF traces also have sharper increase towards the end of the breath holding, e.g. as shown in graphs 1805 and 1807, in comparison to the CBV, e.g. as shown in graphs 1802 and 1804. This points to stressing to the limit of cerebral autoregulation where blood vessels cannot further expand to accommodate the blood pressure, thus the sudden increase in CBF towards the end of the breath holding.

### Computational Devices

The techniques described above may be implemented using one or more computing devices. FIG. 19 illustrates components of an example computing device that may be used, e.g., to implement blocks of process 1000 of FIG. 10, process 1100 of FIG. 11, or process 1200 of FIG. 12, respectively. Note that such a computing device may be part of a headset comprising one or more light sources and/or one or more light detectors (e.g., the computing device may be disposed on a portion of the headset or headband), or may be communicatively coupled to the headset (e.g., via a wireless communication channel, such as BLUETOOTH).

In FIG. 19, the computing device(s) 1950 includes one or more processors 1960 (e.g., microprocessors), a non-transitory computer readable medium (CRM) 1970 in communication with the processor(s) 1960, and one or more displays 1980 also in communication with processor(s) 1960. Processor(s) 1960 is in electronic communication with CRM 1970 (e.g., memory). Processor(s) 1960 is also in electronic communication with display(s) 1980, e.g., to display image data, text, etc. on display 1980. Processor(s) 1960 may retrieve and execute instructions stored on the CRM 1970 to perform one or more functions described above. For example, processor(s) 1960 may execute instructions to perform one or more operations to analyze collected data (e.g., light reflection/absorption data). The CRM (e.g., memory) 1970 can store instructions for performing one or more functions of the described above. These instructions may be executable by processor(s) 1960. CRM 1970 can also store raw images, e.g., speckle images, or the like.

### Example Embodiments

Embodiment 1: A headset for generating cerebral blood metric data, the headset comprising: a headband configured to be worn on a head; and a laser coupled to the headband and configured to emit light into a brain within a skull of the head; a light detector coupled to the headband and configured to generate information indicative of light reflected from one or more structures within the brain, wherein the light detector is configured to contact or be within 5 mm of a scalp of the skull; and a power source coupled to the headband and in electrical communication with the laser.

Embodiment 2: The headset of embodiment 1, wherein the headset is configured to be able to adjust a distance between the laser and the light detector.

Embodiment 3: The headset of embodiment 1, wherein the laser is a continuous laser.

Embodiment 4: The headset of any one of the embodiments 1-3, wherein the laser is configured to emit light in a near-infrared or infrared wavelength.

Embodiment 5: The headset of embodiment 1, wherein the headband comprises one or more straps.

Embodiment 6: The headset of embodiment 1, wherein the headband is configured to encircle the head.

Embodiment 7: The headset of embodiment 1, wherein the headband is configured to place the light detector in contact with a region of a scalp of the head.

Embodiment 8: The headset of embodiment 7, wherein the headband is further configured to apply pressure to the scalp at the region of the scalp in contact with the light detector.

Embodiment 9: The headset of any one of embodiments 1-8, further comprising one or more processors configured to: cause, using the laser, light to be emitted into the brain; obtain, using the light detector, the information indicative of the reflected light from one more structures within the brain; and based on the obtained information, determine one or more cerebral blood metrics.

Embodiment 10: The headset of embodiment 9, wherein the one or more cerebral blood metrics comprises one or more of a cerebral blood flow, a cerebral blood volume, or a heart rate.

Embodiment 11: The headset of embodiment 9, wherein the one or more cerebral blood metrics comprises one or more of a cerebral blood flow, a cerebral blood volume, or a heart rate in a region of the brain.

Embodiment 12: The headset of embodiment 1, wherein the light detector is configured to probe a region of the brain.

Embodiment 13: The headset of embodiment 1, wherein the laser, the light detector, and the power source form a first channel.

Embodiment 14: The headset of embodiment 13, further comprising one or more additional channels, wherein each additional channel comprises an additional light detector and wherein the additional light detectors of the additional channels are positioned to probe different regions of the brain.

Embodiment 15: The headset of embodiment 14, wherein at least one of the additional channels comprises the laser and the power source of the first channel.

Embodiment 16: The headset of embodiment 1, wherein the light detector comprises a plurality of sensor segments.

Embodiment 17: The headset of embodiment 16, wherein the sensor segments are configured to probe different regions at different depths of the brain.

Embodiment 18: The headset of embodiment 17, wherein each of the sensor segments comprises a complementary metal-oxide-semiconductor sensor.

Embodiment 19: A multi-channel headset comprising: a headband configured to be worn on a head during operation; and a plurality of channels coupled to the headband, each channel comprising: a laser configured to emit light into a brain within a skull of the head; a light detector configured to generate information indicative of light emitted by the laser and reflected by one or more structures within the brain; and a power source in electrical communication with the laser; wherein the light detectors of the channels are configured to probe different regions of the brain.

Embodiment 20: The multi-channel headset of embodiment 19, wherein at least two of the channels have light detectors configured to receive light from the same laser.

Embodiment 21: An apparatus comprising: a circuit board; one or more processors attached to the circuit board; a laser diode attached to the circuit board; a light detector in electrical communication with the one or more processors; a light block located between the laser diode and the light detector; and a power source in electrical communication with the laser diode.

Embodiment 22: The apparatus of embodiment 21, further comprising a wireless receiver for transmitting wireless signals, the wireless receiver in electrical communication with the one or more processors.

Embodiment 23: The apparatus of embodiment 21, wherein the light block has a height in a range of 3 mm and 1 cm and a length in a range of 2 cm and 5 cm.

Embodiment 24: The apparatus of any one of embodiments 21-23, wherein the one or more processors comprise one or both of an application-specific integrated circuit and a programmable logic device.

Embodiment 25: The apparatus of embodiment 24, wherein the programmable logic device is a field-programmable gate array.

Embodiment 26: The apparatus of any of embodiment 21-25, wherein the circuit board and/or the light detector is made of a flexible material.

Embodiment 27: The apparatus of embodiment 21, wherein the light detector has a length in a range of 15 mm and 25 mm.

Embodiment 28: The apparatus of embodiment 21, wherein the light detector is positioned such that its length is perpendicular to a surface of the light block.

Embodiment 29: The apparatus of any one of embodiments 21-28, wherein the light detector comprises a plurality of complementary metal-oxide-semiconductor sensors.

Embodiment 30: The apparatus of any one of embodiments 21-29, wherein the light detector comprises a plurality of sensor segments.

Embodiment 31: The apparatus of embodiment 30, wherein the sensor segments are configured to probe different regions at different depths.

Embodiment 32: The apparatus of any one of embodiments 21-32, wherein the one or more processors are configured to: cause, using the laser diode, light to be emitted into a brain within a skull of a head of a user; obtain, using the light detector, information indicative of light reflected from one more structures within the brain; and based on the obtained information, determine one or more cerebral blood metrics.

Embodiment 33: The apparatus of embodiment 32, wherein the obtained information comprises a speckle pattern obtained using images captured by the light detector.

Embodiment 34: The apparatus of embodiment 32, wherein a portion of the obtained information spans a time period during which the user was holding their breath.

Embodiment 35: The apparatus of embodiment 33, wherein the one or more cerebral blood metrics comprise a cerebral blood flow determined based on the speckle pattern.

Embodiment 36: The apparatus of any one of embodiments 21-35, further comprising a headband configured for attachment to a head, wherein the circuit board is attached to the headband such that the light detector is configured to contact or be in close proximity to a scalp of the head.

Embodiment 37: The apparatus of embodiment 36, further comprising one or more additional circuit boards attached to the headband, each circuit board comprising an additional laser diode, an additional light detector, an additional light block, and an additional power source.

Embodiment 38: A method of determining one or more cerebral blood metrics, the method comprising: causing, using a laser, light to be emitted into a brain within a skull of a head of a user; obtaining, using a light detector, information indicative of light reflected from one more structures within the brain; and based on the obtained information, determining one or more cerebral blood metrics as a function of time.

Embodiment 39: The method of embodiment 38, wherein the laser and the light detector are disposed on a headset worn by the user.

Embodiment 40: The method of embodiment 38, wherein the obtained information comprises a plurality of speckle images.

Embodiment 41: The method of embodiment 40, further comprising: normalizing each speckle image based on a first set of the speckle images acquired during a first time period; calculating a speckle contrast of each normalized speckle image; and adjusting the speckle contrast to account for noise.

Embodiment 42: The method of any one of embodiments 40-41, further comprising calculating a cerebral blood flow from the adjusted speckle contrast of each image of a second set of the speckle images acquired during a second time period in which the user was holding their breath.

Embodiment 43: The method of any one of embodiments 40-42, further comprising: calculating a plurality of cerebral blood flow values over time from the adjusted speckle contrast of a second set of the speckle images acquired during a second time period in which the user was holding their breath; and calculating a heart rate from a period in the cerebral blood flow values over time.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

1. A headband configured to be worn on a head; and
a laser coupled to the headband and configured to emit light into a brain within a skull of the head;
a light detector coupled to the headband and configured to generate information indicative of light reflected from one or more structures within the brain, wherein the light detector is configured to contact or be within 5 mm of a scalp of the skull; and
a power source coupled to the headband and in electrical communication with the laser.

2. The headband of claim 1, wherein a distance between the laser and the light detector is adjustable by changing position of the laser and/or the light detector on the headband.

3. The headband of claim 1, wherein the laser is a continuous laser and/or the laser is configured to emit light in a near-infrared or infrared wavelength.

4. (canceled)

5. The headband of claim 1, wherein the headband comprises one or more straps and/or is configured to encircle the head.

6. (canceled)

7. The headband of claim 1, wherein the headband is configured to place the light detector in contact with, and apply a pressure to, a region of the scalp of the head.

8. (canceled)

9. The headband of claim 1, further comprising one or more processors configured to:
cause, using the laser, light to be emitted into the brain;
obtain, using the light detector, the information indicative of the reflected light from one more structures within the brain; and
based on the obtained information, determine one or more cerebral blood metrics.

10. The headband of claim 9, wherein the one or more cerebral blood metrics comprises one or more of a cerebral blood flow, a cerebral blood volume, or a heart rate.

11-12. (canceled)

13. The headband of claim 1, wherein the laser, the light detector, and the power source form a first channel and further comprising one or more additional channels, wherein each additional channel comprises an additional light detector and wherein the light detectors of the first channel and the additional channels are configured to probe different regions of the brain.

14-15. (canceled)

16. The headband of claim 1, wherein the light detector comprises a plurality of sensor segments at different distances from the laser.

17. The headband of claim 16, wherein the sensor segments are configured to probe different regions at different depths of the brain.

18. (canceled)

19. A multi-channel headset comprising:
a headband configured to be worn on a head during operation; and
a plurality of channels coupled to the headband, each channel comprising:
a laser configured to emit light into a brain within a skull of the head;
a light detector configured to generate information indicative of light emitted by the laser and reflected by one or more structures within the brain; and
a power source in electrical communication with the laser;
wherein the light detectors of the channels are configured to probe different regions of the brain.

20. The multi-channel headset of claim 19, wherein at least two of the channels have light detectors configured to receive light from the same laser.

21. An apparatus comprising:
a circuit board;
one or more processors attached to the circuit board;
a laser diode attached to the circuit board;
a light detector in electrical communication with the one or more processors; and
a light block located between the laser diode and the light detector.

22. The apparatus of claim 21, further comprising (i) a power source in electrical communication with the laser diode and/or (ii) a wireless transmitter for transmitting wireless signals, the wireless transmitter in electrical communication with the one or more processors.

23-24. (canceled)

25. The apparatus of claim 21, wherein the one or more processors comprise one or both of an application-specific integrated circuit and a programmable logic device.

26. (canceled)

27. The apparatus of claim 21, wherein the circuit board and/or the light detector is made of a flexible material.

28-29. (canceled)

30. The apparatus of claim 21, wherein the light detector comprises a plurality of complementary metal-oxide-semiconductor sensors.

31. The apparatus of claim 21, wherein the light detector comprises a plurality of sensor segments configured to probe different regions at different depths.

32. (canceled)

33. The apparatus of claim 21, wherein the one or more processors are configured to:
cause, using the laser diode, light to be emitted into a brain within a skull of a head of a user;
obtain, using the light detector, information indicative of light reflected from one more structures within the brain; and
based on the obtained information, determine one or more cerebral blood metrics.

34-38. (canceled)

39. A method of determining one or more cerebral blood metrics, the method comprising:
causing, using a laser, light to be emitted into a brain within a skull of a head of a user;
obtaining, using a light detector, information indicative of light reflected from one more structures within the brain; and
based on the obtained information, determining one or more cerebral blood metrics as a function of time.

40. (canceled)

41. The method of claim 39, wherein the obtained information comprises a plurality of speckle images.

42. The method of claim 41, further comprising:
normalizing each speckle image based on a first set of the speckle images acquired during a first time period;
calculating a speckle contrast of each normalized speckle image; and
adjusting the speckle contrast to account for noise.

43. The method of claim 42, further comprising calculating a cerebral blood flow from the adjusted speckle contrast of each image of a second set of the speckle images acquired during a second time period in which the user was holding their breath.

44. The method of claim 42, further comprising:
calculating a plurality of cerebral blood flow values over time from the adjusted speckle contrast of a second set of the speckle images acquired during a second time period in which the user was holding their breath; and
calculating a heart rate from a period in the cerebral blood flow values over time.
